(19) United States
(12) Patent Application Publication
(10) Pub. No.: US 2026/0166198 A1
(43) Pub. Date: Jun. 18, 2026

(54) MEW TISSUE SCAFFOLD

(71) Applicant: The University of Western Australia, Nedlands (AU)

(72) Inventors: Michael John Vernon, City Beach (AU); Maria Elena Juan Pardo, Manning (AU); Christopher Lamb, Treeby (AU)

(21) Appl. No.: 19/122,896

(22) PCT Filed: Oct. 20, 2023
(86) PCT No.: PCT/AU2023/051054

§ 371 Date: Apr. 21, 2025

(30) Foreign Application Priority Data
Oct. 21, 2022 (AU) 2022903120

(51) Int. Cl. A61L 27/14; A61F 2/24

(52) U.S. Cl. CPC A61L 27/14; A61F 2/2415; A61F 2/2418; A61F 2250/0023; A61L 2430/20

(57) ABSTRACT

A melt electrowritten soft tissue scaffold, comprising: a first region having one or more sets of fibres and a second region having one or more sets of fibres; and an interface region joining the first region and the second region, the interface region being electrowritten along with the first and second regions in a continuous printing path, such that fibres in the interface region each join between a respective pair of fibres in the first and second regions.

### TECHNICAL FIELD

This disclosure relates generally to implantable devices or scaffolds, such as those for engineered heart valves. More particularly the invention relates to interfaces between structural regions and gradients within the scaffolds.

### INCORPORATION OF REFERENCE

The inventors previously developed a novel method of providing a scaffold or an implant using melt electrowriting (MEW), as disclosed in patent cooperation treaty application PCT/AU2020/210877, the contents of which are incorporated herein by reference.

### BACKGROUND ART

Biological tissues are complex, multi-phasic, heterogeneous, hierarchical structures, that present exquisite properties ideally suited for their function. Moreover, tissue interfaces play the critical role of not only physically connecting heterogeneous regions but also exhibiting graded structural, cellular, and mechanical features, and thus critically contributing to the overall functionality of the tissue or organ. Accordingly, when attempting to biofabricate scaffolds for these tissues, the interfaces represent a pivotal, yet challenging piece of the puzzle.

The majority of research surrounding engineering of tissue interfaces relates to soft-hard tissue interfaces in areas such as ligaments and tendons, cartilages, as well as dental and craniomaxillofacial implants. In these applications, heterogeneous scaffolds have been achieved through advanced manufacturing techniques, such as multi-axial extrusion, varying degrees of crosslinking, two step phase separation, multi-material bioinks, and through controlled spatial deposition of biomaterials with advanced three-dimensional (3D) printing technologies.

Fibrous scaffolds and in particular electrospun meshes have been extensively investigated in the field of soft tissue engineering for applications such as skin, neural, vascular or cardiac tissue. However, there is a lacking body of comparable research towards interfacial and gradient design. Interfacing between regions has been achieved mostly on a layer-by-layer basis, by either altering print parameters or solution concentration during the fabrication process, crosslinking afterwards, or individual layer-by-layer assembly. The absence of interface complexity and gradient structures for electrospun scaffolds could be due to the lack of precise control over fibre orientation when manufacturing with this technology.

MEW is a highly precise additive manufacturing technique capable of printing complex fibrous scaffold constructs at sub-micron resolutions. The complexity and resolution characteristics of MEW have been beneficial for producing functional, biomimetic, soft tissue scaffolds for skin, nerves, myocardium, cartilage and aortic heart valves. Similar to electrospinning, interfacing on a layer-by-layer basis has been shown with MEW. More recently, the ability to fabricate heterogeneous fibrous structures within a layer using MEW has been demonstrated, marking a notable step forward in the field of fibrous scaffold fabrication. Despite this, the concept of heterogeneous MEW printing is still in its infancy, and as a result, the design of the interface between heterogeneous regions has not been a primary focus of previous work.

It is to be understood that, if any prior art is referred to herein, such reference does not constitute an admission that the prior art forms a part of the common general knowledge in the art, in Australia or any other country.

### SUMMARY

In a first aspect, herein disclosed is a melt electrowritten scaffold. The scaffold comprises a first region having one or more sets of fibres and a second region having one or more sets of fibres. The first region and the second region are joined by an interface region, the interface region being electrowritten along with the first and second regions along a continuous printing path within the same layer, such that fibres in the interface region each join between a respective pair of fibres in the first and second regions.

The scaffold may be used for the engineering of a biodegradable or non-biodegradable implantable device.

The first and second regions can be heterogeneous in that they differ from each other in one or more spatial parameters.

The path for at least one of the fibres within the interface region can be defined manually or defined by a mathematical function.

The function can be such that the fibre has a complex shape as it transitions from the one of the first or second regions through the interface region and to the other one of the first or second regions.

The second region can have a higher porosity than the first region.

The higher porosity in the second region can be at least partially formed by joining every two or more adjacent fibres in the first region as they transition to the second region.

The higher porosity in the second region can be at least partially formed by causing the fibres to fan outwards as they transition to the second region.

The higher porosity in the second regions can be at least partially formed by setting the continuous printing path to cause an additional set of fibres to be deposited offset from the other fibres in the first region by a distance which is less than the size of a pore size.

The first region or the second region, or both, can include a first set of fibres arranged approximately in parallel to each other and a second set of fibres arranged approximately in parallel to each other, the second set of fibres being arranged at an angle and preferably transversely relative to the first set of fibres, each fibre in the second set of fibres having a serpentine arrangement having defined troughs and peaks.

A fibre arrangement in the interface region can be at least partially biomimetic. For example, the fibre arrangement can be based on a simplified form of collagen fibre organisation observed in an interfacial region of a biological sample of the soft tissue. The collagen fibre organisation can be determined by an orientation analysis.

The scaffold can have a plurality of layers of fibres.

A portion of the scaffold can have a different number of layers than another portion of the scaffold. The scaffold can be a heart valve scaffold. The first region can be a leaflet and the second region can be an inter-leaflet triangle.

In another aspect, herein disclosed is a method of providing a melt electrowritten soft tissue scaffold having at least two structurally heterogeneous regions and an interface region there between, including providing a printing path along which polymer melt material is to be continuously extruded during a melt electrowriting process to form the heterogeneous regions and the interface region.

The method can include causing the continuous print path to be piece-wise defined, such that it is defined by different functions in the heterogeneous regions and in the interface region.

In another aspect, herein disclosed is also a method for designing a MEW scaffold for a soft tissue having a plurality of structural regions. The method includes imaging the soft tissue using an imaging modality which is capable of imaging of collagen fibre structures within the soft tissue, identifying an interface region from the structural regions, applying orientation analysis to at least image(s) from the imaging step showing the interface region, determining a simplified form of a fibre distribution observed in the orientation analysis, configuring a print path for an interface gradient region of the scaffold corresponding to the interfacial region of the soft tissue, on the basis of the simplified form of the fibre distribution

Function or functions which define the continuous print path as it traverses through the interface gradient region is/are a mathematical function, preferably providing a curved or serpentine shape.

The present invention has scaffold-based biomedical applications, as native tissues are very rarely homogenous i.e. gradients are present throughout all types of tissue.

In an aspect of the present invention, the scaffold comprises gradients of the fibres comprising the scaffold. By gradient it is meant a gradual spatial change in properties. This change can be with respect to geometric features (e.g. size, thickness, shape, density, orientation), mechanical properties (stiffness, elasticity), composition (cell/tissue type), chemistry (pH) or more. The present scaffolds may be used to create multiple gradient properties in a single scaffold.

For example, herein disclosed is a melt electrowritten scaffold comprising gradient porosities. The terms “gradient porosity” or “porosity gradient” refers to a change in size and/or shape of pores in the scaffold across regions of the scaffold. The size of the pores may gradually increase or decrease in either one direction, or two directions spatially across the scaffold. The gradient change of porosity is determined by a mathematical function, wherein the shape of the pore within the scaffold can be, for example, either rectangular, diamond or serpentine.

Preferably, the scaffold comprises a plurality of layers of fibres. The plurality of layers of fibres may comprise gradients of geometric features and/or mechanical properties to form a gradient scaffold. Each fibre of the scaffold may comprise gradients of geometric features and/or mechanical properties to form a gradient scaffold. For example, the fibres may be thicker, denser and/or stiffer in one region, with a gradient to thinner, less dense and/or more flexible in another region. Function or functions which define the continuous print path as it traverses through the gradient region(s) is/are a mathematical function, defining the geometric features and/or mechanical properties of the fibres in the region.

The scaffold gradient design may be used in either all or some of the regions of the scaffold.

Gradient functions may be generated for heart valve applications, incorporating patient-specific anatomy of the heart valve inspired by the orientation, distribution and densities of native collagen fibres.

The scaffold fibres that form the porosity gradient may be purposely aligned either parallel or orthogonal to the interface between two regions. In the context of heart valves, this would mean aligning them with the interfaces between the leaflet and the interleaflet triangle/commissure/annulus.

The porosities in the gradient may be arranged to be smaller in the region of the commissures, and larger in the belly of the leaflet, or vice versa, to reflect regions of higher loading.

The fibre density gradients may be arranged to be higher in the region of the commissures and belly of the leaflet, above the annulus, and smaller inside the leaflet, or vice versa, to reflect regions of higher loading.

Any of the above types of gradients (geometric features, mechanical properties, composition, chemistry etc) may be combined or applied individually to design melt electrowritten scaffolds for heart valves or other tissues.

### BRIEF DESCRIPTION OF THE DRAWINGS

Embodiments will now be described by way of example only, with reference to the accompanying drawings.

FIG. 1 schematically illustrates parts of a MEW device.

FIG. 2 is a digital microscope image of an example MEW scaffold with two heterogeneous regions and a continuous interface between the heterogeneous regions.

FIG. 3-1 is a schematic depiction of scaffold having constant porosity throughout.

FIG. 3-2 is a schematic depiction of a scaffold having an arrangement where pairs of fibres from the scaffold of FIG. 3-1 have been joined to double the pore size.

FIG. 3-3 is a schematic depiction of “halving” of a scaffold, whereby another fibre layer is added to the scaffold in order to half the pore size in a section of the scaffold.

FIG. 3-4 is a schematic depiction of “fanning” of a scaffold, whereby fibres of the second (right hand side) section are fanned out so as to gradually increase the pore size in the section.

FIG. 3-5 an example image of a MEW fabricated device in which “halving” is performed in a section of the scaffold.

FIG. 3-6 is an example scaffold having a section in which fibres fan to decrease the pore size.

FIG. 3-7 is an example scaffold having a section in which fibres fan to increase the pore size.

FIG. 4-1 includes schematic depictions of the overlap, suture, and continuous interfaces.

FIG. 4-2 shown digital microscope (top) and SEM (bottom) images of MEW PCL scaffolds with 1 mm pore square patterns and 0.5 mm pore diamond patterns interfaced with the overlap, suture and continuous methods, scale bars=1 mm.

FIGS. 5-1 to 5-3 show images obtained during uni-axial tensile testing of bi-phasic MEW scaffolds interfaced using either the overlap, suture, or continuous technique. FIG. 5-1 shows time-lapse images through tensile testing of a continuously interfaced 1 mm pore serpentine and 0.5 mm pore square scaffold. Scale bars=5 mm. FIG. 5-2 shows representative stress-strain plot of continuously interfaced scaffold with labels corresponding to regions of i) elastic deformation, ii) plastic deformation, and iii) scaffold rupture. FIG. 5-3 shows combined representative stress-strain plots for the serpentine-square scaffolds with each type of interface and control scaffolds.

FIG. 5-4 shows Young's modulus, yield strength and ultimate tensile strength for each type of bi-phasic scaffold with interface method and control scaffolds (average over n=3, error bars represent one standard deviation, significant differences were assessed using one-way ANOVA with Tukey's multiple comparisons test, where statistical significances are shown over a singular column this data had at least that level of significant difference when compared to every other column on the graph, *p≤0.05, **p≤0.01, ***p≤0.001, ****p≤0.0001).

FIG. 6-1 shows images of clamping distances of 2.5 mm, 4.5 mm, 6.5 mm applied to the MEW scaffold for testing uni-axial tensile properties.

FIG. 6-2 show the representative stress versus strain plots of applying strain to continuously interfaced serpentine-square scaffold, respectively at clamping distances 2.5 mm, 4.5 mm, 6.5 mm.

FIG. 6-3 shows the Young's Modulus measured with strain applied at different clamping distances.

FIG. 6-4 shows the yield strength measured with strain applied at different clamping distances.

FIG. 6-5 shows the ultimate tensile strength measured with strain applied at different clamping distances.

FIG. 7-1 schematically depicts a flexural testing apparatus with θ representing the bending angle measured.

FIG. 7-2 is a close-up view of a bi-phasic scaffold displaying the location of the pivot line, scale bar=1 mm.

FIG. 7-3 is an image of testing of multi-phasic scaffold with serpentine pattern, scale bar=10 mm.

FIG. 7-4 depicts flexural stiffness data (average over n=3, error bars represent standard deviation, two-way ANOVA with Tukey's multiple comparisons test resulted in p<0.0001 for effect of pattern, p=0.0002 for effect of interface and non-significant differences between individual means).

FIG. 8-1 is an example graphical user interface for generating continuous and spatially heterogeneous G-codes.

FIG. 8-2 is an example scaffold designed using the graphical user interface (GUI).

FIG. 8-3 is another example scaffold designed using the graphical user interface (GUI).

FIG. 8-4 shows digital microscope images, including an image of a MEW PCL scaffold having the design shown in FIG. 8-2, a high-resolution image of 1 mm pore auxetic star pattern in section “A” and a high resolution of the fanning interface showing continuous transition from 1 mm pores to 2 mm pores.

FIG. 8-5 shows digital microscope images, including an image of a of MEW PCL scaffold having the design shown in FIG. 8-3, a high-resolution image of 1 mm pore diagonal serpentine pattern in section ‘A”, and a high resolution of the fanning interface showing continuous transition from 1 mm pore to 2 mm pores. scale bars=2 mm.

FIG. 9-1 is an image showing regions of an aortic valve.

FIG. 9-2 depicts the 3D reconstructed valve architecture and the sectioning planes, as viewed from the i) coronal (external), ii) sagittal, iii) axial, and iv) coronal (internal) directions.

FIG. 9-3 is a second harmonic generation (SHG) image of an aortic valve, showing the commissure and interface with the beginning portions of adjacent leaflets.

FIG. 9-4 is an SHG image of a slice of the aortic valve sample showing the commissure.

FIG. 9-5 is an SHG image of a slice of the aortic valve sample showing the interleaflet triangle.

FIG. 10 is a schematic depiction of a hybrid immersion fixation arrangement to fix a porcine aortic valve tissue sample.

FIG. 11-1 is a second harmonic generation (SHG) image of aortic valve commissure, displaying the location of the imaging plane selected for correlative focused ion beam scanning electron microscopy (FIBSEM), scale bar=1 mm.

FIG. 11-2 is a low magnification FIBSEM image taken in the circumferential direction displaying the aortic valve fibro-cellular microstructure in the upper regions of the commissure, scale bar=10 μm.

FIG. 11-3 is a magnified image of the region bounded by the dashed line box in FIG. 11-2, showing individual collagen fibre cross sections travelling in the circumferential (C) direction, and longitudinal fibre sections running in the radial (R) and longitudinal (L) direction, scale bar=1 μm.

FIG. 12-1 depicts a colour-mapped orientation analysis of an SEG image of the inter-leaflet triangle region, scale bars=0.5 mm.

FIG. 12-2 is a simplified, schematic representation of collagen fibre orientation in the inter-leaflet triangle.

FIG. 12-3 depicts the resulting MEW scaffold and a colour-mapped orientation analysis of interfacial region, scale bars=2 mm.

FIG. 12-4 shows overlayed orientation distribution from an inter-leaflet triangle tissue sample and MEW scaffold.

FIG. 12-5 shows the resulting MEW scaffold labelled with inputted G-code showing design parameters.

FIG. 12-6 shows snap shots of the G-code path with corresponding time stamps at time=1, 2, 6, 29, and 74 seconds after the start time, to display the continuous print path at these times.

FIG. 12-7 is an SEM image showing close-up view of continuous fibre interface employing joining technique where fibres have fused upon joining paths, scale bar=100 μm.

FIG. 13-1 is an image of a bi-axial tensile testing set up for applying strain in circumferential and longitudinal testing directions, scale bar=5 mm.

FIG. 13-2 are result graphs for the measured Young's modulus, yield strength and yield strain for the circumferential and longitudinal tests.

FIG. 13-3 depicts the hysteresis behaviour from cyclical testing at constant strain showing representative stress-strain plots in both directions.

FIG. 13-4 depicts quantification of hysteresis shown by the area under the unloading curve divided by the area under the loading curve for the same cycle.

FIG. 13-5 depicts a representative relaxation behaviour after four incremental increases in strain (2.5% and 5% in the longitudinal and circumferential direction respectively), held for 1000 seconds, followed by quantification of relaxation percentage for each step (all data shows average over n=3, error bars represent one standard deviation, significant differences were assessed using parametric ratio paired t tests, *p≤0.05, **p≤0.01, ***p≤0.001).

FIG. 13-6 depicts the relaxation percentage versus the number of strain steps applied.

FIG. 14-1 is a conceptual illustration of an example of a sinusoidal interface.

FIG. 14-2 is a conceptual illustration of an example of an angled interface.

FIG. 14-3 is a conceptual illustration of an example of an arrow head shaped interface.

FIG. 14-4 illustrates an example scaffold schematic for a layer, showing a sinusoidal interface transitioning between a region with a rectangular grid pattern and a region where the fibres are arranged in an “auxetic stars” pattern.

FIG. 14-5 illustrates an example scaffold schematic for a layer, showing an “angled” interface transitioning between a region with a rectangular grid pattern and a region where the fibres are arranged in an “auxetic stars” pattern.

FIG. 14-6 illustrates an example scaffold schematic for a layer, showing an arrow or arrowhead shaped interface transitioning between a region with a rectangular grid pattern and a region where the fibres are arranged in an “auxetic stars” pattern.

FIG. 15-1 show the parameters used to generate the scaffold layer schematic shown in FIG. 14-4.

FIG. 15-2 show the parameters used to generate the scaffold layer schematic shown in FIG. 14-5.

FIG. 15-3 show the parameters used to generate the scaffold layer schematic shown in FIG. 14-6.

FIG. 16 is an schematic showing exemplary gradient porosity scaffolds and the underlying mathematical function governing the gradient porosities, where the value of the initial pore size (p0), and the gradient coefficient (m) can be varied. The gradient function has been applied to three different patterns (rectangular, diamond or serpentine) and affects the pore-dimension either parallel or orthogonal to the direction of the gradient (which is applied horizontally, left to right).

FIG. 17 is an exemplary preview of parallel and orthogonal gradient porosity scaffolds for rectangular, diamond or serpentine patterns. All scaffolds have dimensions of 25×25 mm, initial pore size (p0) of 0.5 mm and gradient coefficient (m) of 0.5, 1.0 or 1.5. Note that in the case of orthogonal gradient porosity scaffolds, the gradient coefficient refers to the steepest gradient line, and the rest of the lines are interpolated between that and flat (zero gradient).

FIG. 18 shows exemplary gradient porosity scaffolds fabricated using melt electrowriting using a 23G needle, 100 kPa pressure, working distance of 3 mm, potential difference of 3.8 kV, print speed of 400 mm/min, syringe temperature of 75° C., needle temperature of 85° C. and bed temperature of 30° C. Each scaffold was made from polycaprolactone, has a dimension of 25×25 mm, with five layers and an average fibre diameter of 20 μm.

FIG. 19 shows local strain mapping of gradient scaffolds undergoing constrained bi-axial tensile testing. Scaffolds were clamped using 15 mm clamps and strained at 1%/s in the horizontal direction to 100% strain, while being fixed in the vertical direction. Images taken at 20% strain. Colour maps show heterogeneous distribution of principal engineering strain calculated using VIC-2D software (Correlated Solutions, USA).

FIG. 20 shows heterogeneous loading throughout different regions of the valve, particularly in regions of centre-belly and commissure. Figure adapted from: Emmert, et al. Science Translational Medicine 10, no. 440 (2018). https://doi.org/10.1126/scitranslmed.aan4587.

FIG. 21 shows exemplary heart valve scaffold design aspects incorporating gradients. A) Design from PCT/AU2020/210877 featuring uniform serpentine amplitude/wavelength throughout, alignment in two directions only: circumferential (blue, left-right) or radial (red, top-bottom) and anisotropic fibre density/porosity (higher density in circumferential, lower density in radial). B) Radial fibres cross orthogonal (or near-orthogonal) to the leaflet-line. C) Circumferential fibres moving parallel to the leaflet-line, to some degree. D) Serpentine wavelength and/or amplitude changes across leaflet. E) Fibre density changes throughout leaflet (in regions of higher loading e.g. the commissure and bottom of leaflet belly).

FIG. 22 is a schematic of an exemplary heart valve scaffold design incorporating gradient porosities.

FIG. 23 is a schematic of a method of combining fibres continuously and gradually between three leaflets. Note lines are drawn straight for simplicity but this could involve serpentines, gradients and/or other patterns.

### DETAILED DESCRIPTION

In the following detailed description, reference is made to accompanying drawings which form a part of the detailed description. The illustrative embodiments described in the detailed description, depicted in the drawings, are not intended to be limiting. Other embodiments may be utilised, and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

Gradient structures are abundant in living tissues and play a critical role in their functionality. The ability to fabricate structures replicating these gradients and interfaces could be beneficial to achieve functional constructs for tissue engineering applications.

Gradient scaffolds have been investigated in the context of tissues such as bone, tendons, vasculature and myocardium, to name a few. These scaffolds have been fabricated using techniques that control properties such as temperature, pH or concentration of materials spatially throughout a material. Fibrous fabrication technologies such as MEW have also been applied to the creation of gradients, however, only for simple rectangular geometries. In addition, gradients scaffolds can be used to create disease or tissue models, such as using gradient stiffness hydrogels to elicit graded cellular responses.

New approaches to manufacturing micro-fibrous scaffolds, such as MEW, may enable more sophisticated interface or gradient designs. MEW has the ability to enable complex and tailorable interface and gradient structures, which could be beneficial for a large range of tissue engineering applications or disease modelling.

Melt electrowriting (MEW) is a highly precise additive manufacturing technology with the potential to leverage specific aspects of the microstructural features observed in native tissues, such as collagen orientation, into the design of complex biomimetic scaffolds. The printing of MEW scaffolds relies on the precise control over mainly five process parameters: temperature, pressure, voltage, working distance and collector speed, resulting in a highly controllable molten polymer jet of micrometric resolution that is deposited onto a collector in a direct-writing mode. However, unlike in other 3D printers, MEW does not allow stopping and starting of extrusion during a print run, as this would disrupt the Taylor cone and cause printing defects. Thus, conventionally, MEW printing is done for each region of similar spatial features in one uninterrupted printing run. Tissues with spatially heterogeneous regions are thus fabricated by printing each region separately and then joining them together, such as by suturing.

Herein disclosed is an inventive method for using MEW to engineer continuous, user-defined, interfaces between separate structural regions of implantable devices, where the interfacial region and the structural regions are printed continuously on each layer. The design strategy for interfacing the different architectures of multi-phasic MEW scaffolds requires careful consideration, as this could impact their ultimate mechanical and biological functionality.

The continuous interface disclosed herein have application in the MEW printing for different implantable devices. An exemplary application is the fabrication of MEW scaffolds for aortic valves.

The complexity and heterogeneity of the aortic valve makes it a good showcase to examine the technical capabilities of MEW in complex scaffold fabrication for soft tissue interfaces. However, it will be understood that the method has applicability to engineering other tissues than a heart valve scaffold.

The average human aortic valve undergoes over 30 million cycles per year, amounting to over 2 billion cycles during a 70-year lifespan. Such remarkable haemodynamic properties are enabled by the combined biomechanical behaviour resulting from each of the structurally distinguishable regions of the valve and the interfaces between them working together. The inventors previously demonstrated MEW scaffolds with mechanical properties similar to those of physiological heart valve leaflets using a bio-inspired design approach. This was enabled by the data available surrounding the relationship between the mechanical properties of the leaflets and collagen recruitment mechanisms during loading.

In one application, the disclosed method is relevant to regions of the heart valve beyond the leaflets, and the interfaces between them, potentially unlocking further applications of MEW to enhance valve functionality. Regions such as the commissures, inter-leaflet triangles, and the interface between them all play crucial roles in the biomechanical behaviour of heart valves.

Heart valves are heterogeneous structures with highly diverse structural and mechanical properties. These gradient properties are not only present within the leaflet of the heart valve but also in the surrounding structures such as the commissure, interleaflet triangle, and annulus, each of which play an important role in the valve function. Thus, in the context of engineering heart valves, the ability to create gradient scaffolds and thereby control the local mechanical properties is highly valuable.

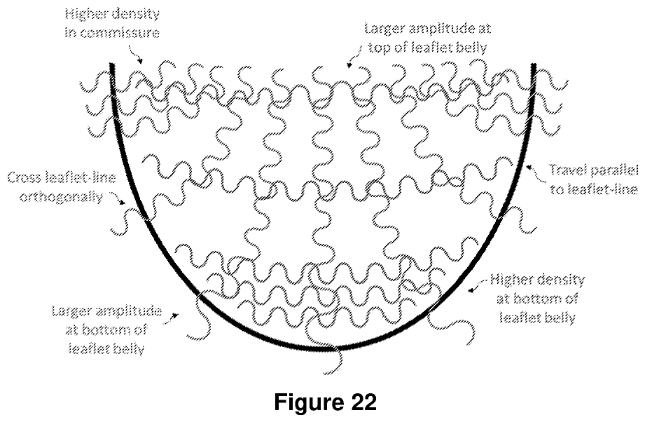

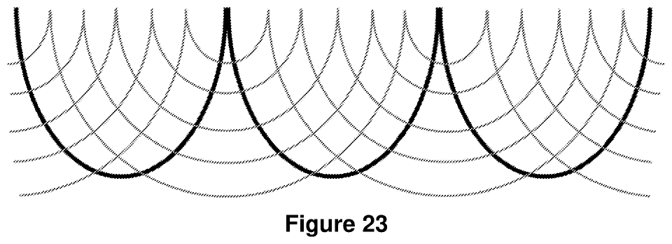

The present invention therefore applies the concept of gradient scaffolds to aspects of fibrous heart valve scaffold design (FIGS. 22, 23).

PCT/AU2020/210877 identified the benefits of joining melt electrowritten fibres continuously between leaflets for improved printing and flexural properties. However, in the previous disclosures the changes in orientations between regions, while being continuous, were abrupt. The present invention provides ways of combining fibres within and between regions gradually and continuously.

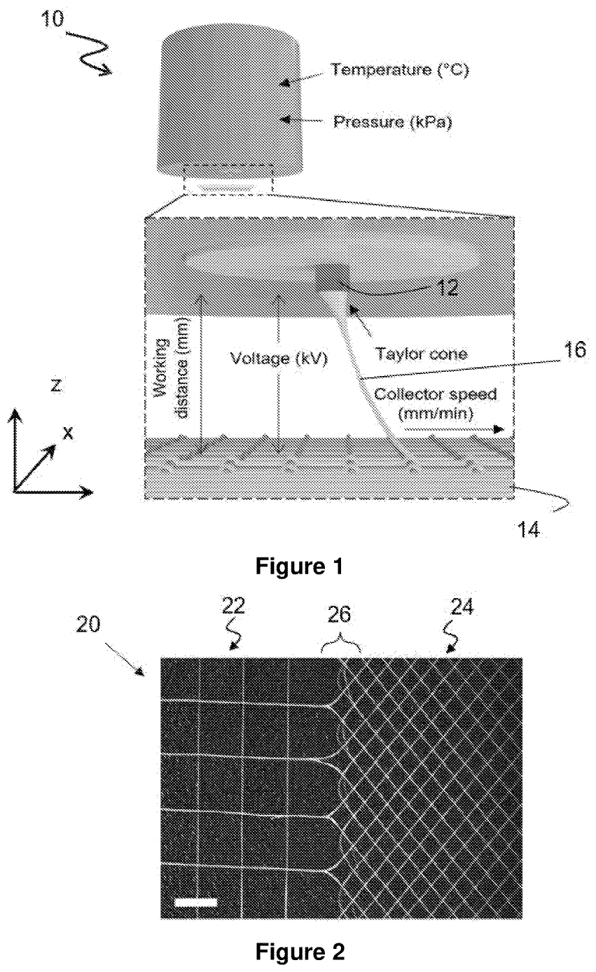

FIG. 1 schematically illustrates parts of a MEW device 10, including the nozzle 12 for extruding a polymer melt onto a collector 14. For clarity, “x”, “y”, and “z” directions mentioned here are used to reference the orientations shown in FIG. 1. The labelling of the directions may be different and does not restrict the scope of the invention.

The nozzle 12 is located above the collector 14 at a working distance (height) which is defined here in the “z” direction. Relative movements in the “x” direction and “y” direction, or a combination, between and of the nozzle 12 and the collector 14, while the nozzle 12 extrudes the polymer, enable the formation of a 2D MEW scaffold over a layer; a multi-layered 3D structure can be created by adding relative movement between nozzle and collector along the “z” direction. The translation of the nozzle 12 in relation to the collector 14, or vice versa, in the “y” direction, defines the translation speed.

The polymer jet extruded from the nozzle 12 is deposited as a fibre 16 onto the collector 14. The fibre 16 follows a printing path which is pre-defined, to form the required structure. The scaffold, at the end of the MEW printing process, will have a plurality of layers of fibres. The number of layers will depend on the MEW setting and the configuration required for the scaffold.

Some but not necessarily all of scaffolds which can be fabricated using the method described herein are heterogeneous. A heterogeneous scaffold includes at least two regions which are spatially heterogeneously structured. The spatial heterogeneity may reside in differences in one or more parameters such as but not limited to pore size in one or more of the dimensions, fibre orientations, and fibre densities, patterns, lengths, etc.

In accordance with the invention, adjacent structural regions are each continuously formed with an interface there between at one or more layers of the scaffold.

FIG. 2 is a digital microscope image of a MEW scaffold layer 20. The layer 20 includes two structural regions 22, 24 and a continuous interfacial region (which may also be referred to simply as “interface”) 26 joining the regions 22, 24. In this example the two regions 22, 24 are spatially heterogeneous. However, the interface described herein can also be used to join regions which are not spatially heterogeneous.

In this example, region 22 has a larger pore size than region 24, and the shapes of the pores in region 22 are more similar to rectangles, whereas the shapes of the pores in region 24 are more similar to diamonds. There is a continuous interface 26 between the heterogeneous regions 22, 24. The exact configurations of the fibres shown are not indicative of essential elements of the invention. Rather they are provided as an example only, serving to illustrate the concept of “continuous interface” as meant in this disclosure, being that the interface joining the two structural regions is printed in continuous fashion along with the structural regions on the same layer.

For example, as can be seen from FIG. 2, starting from region 22, a fibre follows a path defining the grid pattern in region 22 but then at the border between region 22 with the interface region 26 deviates to follow an interface path governed by an interface function when the fibre enters the interfacial region 26. At the border between interfacial region 26 and region 24 the fibre again deviates from its current path (being the interface path) to follow a path defining the diamond pattern in region 24. This deviation, between paths defining regional patterns and interface paths, occurs throughout the printing to print the entire scaffold layer in one continuous print. The overall printing path therefore remains continuous, but at borders of the regions with the interface region, switches to follow different paths respectively defined for the various regions. This arrangement is in contrast to conventional MEW printing, where the different structural regions are printed separately to avoid deviations in print paths intended for the scaffold. The regions are then joined together, typically either by suturing or by overlapping, or by a combination of both.

In the present method, during printing, for each layer, fibres of the layer are formed by depositing the polymer along a printing path which traverses a number of times across the layer 20 until the layer is formed, and the MEW printer then continues to form further layer(s). Printing for the further layers may follow the same print path as the first layer or may follow different print paths if the scaffold to be fabricated is configured to have variations across the layers (i.e., across the thickness of the sheet along the “z” dimension).

The printing path may also be considered to include a number of back and forth passes. The passes may include any or a combination of: one or more passes across a full or partial extent of a width (e.g., along the “y” direction) of the layer; one or more passes across a full or partial extent of a height (e.g., along the “x” direction) of the layer; one or more passes across a full or partial extent of an oblique direction of the layer.

More generally, the printing path will be determined by the MEW printing algorithm (e.g., written in G-code) defining the device (e.g., scaffold) being fabricated. A “continuous print path” here means a path along which the polymer is continuously extruded. The passes for printing the entire sheet will be part of a continuous print path. The print path may include both curved and straight segments, again depending on what is being fabricated.

Thus, a continuous print path will go through different regions. To facilitate this, the algorithm defining the paths for different regions will switch between these paths as the fibre encounters a border between two regions, in order to form one continuous printing path which transitions between the two regions in a layer without the MEW printing process for the layer being paused.

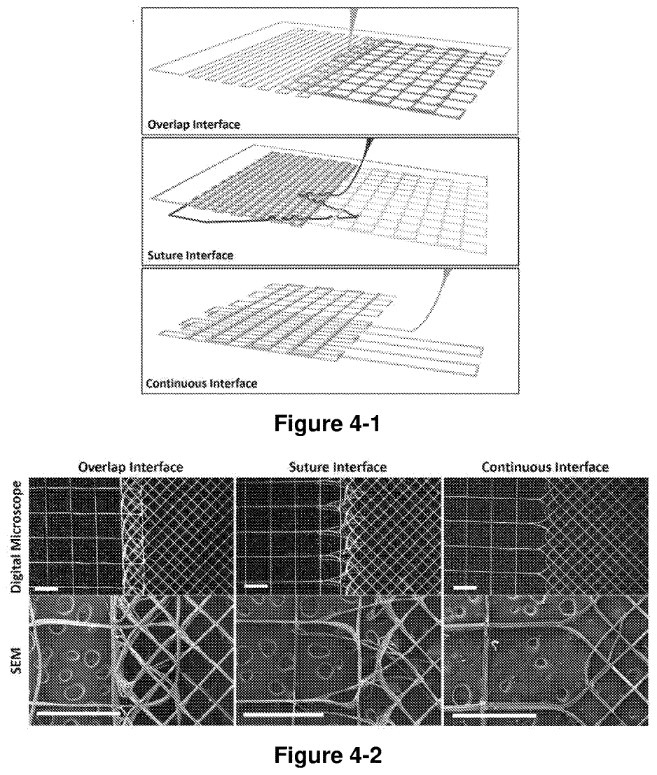

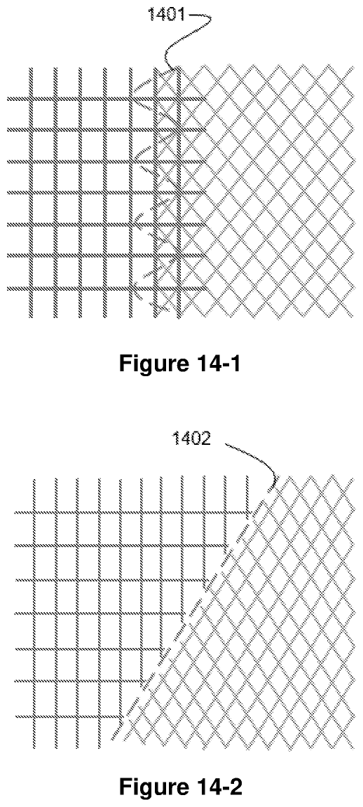

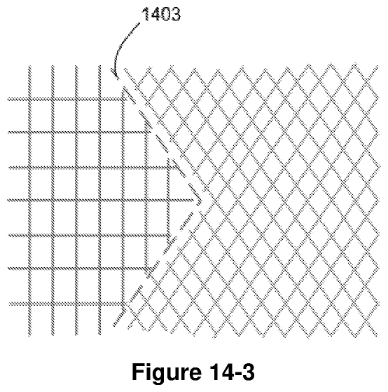

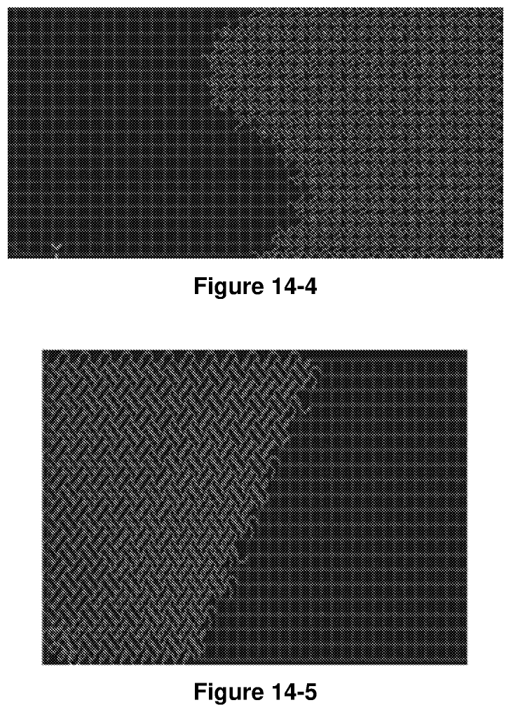

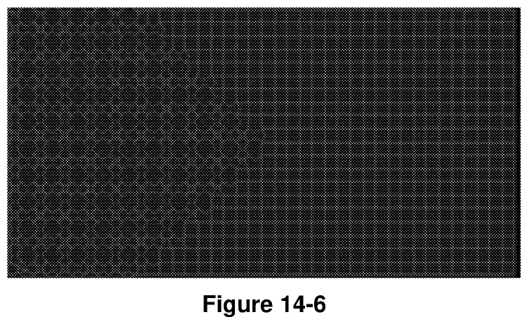

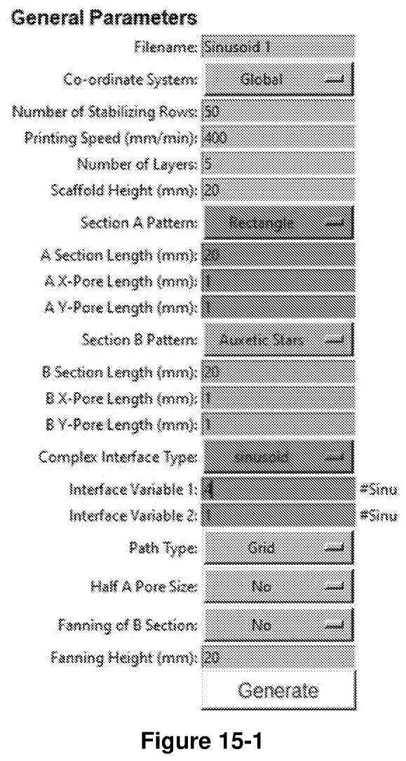

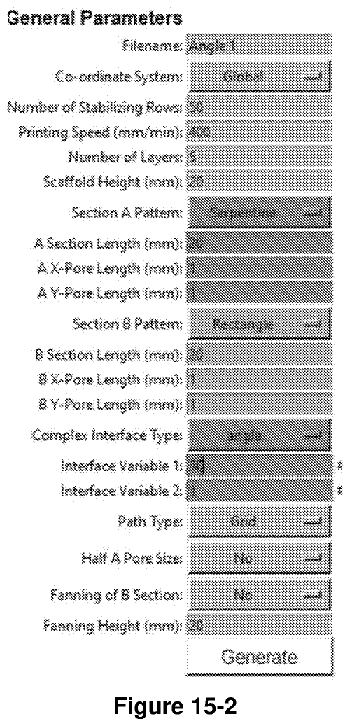

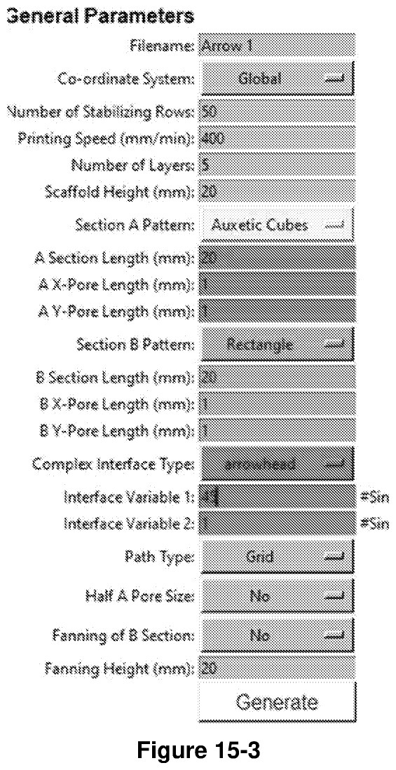

In some embodiments, the print paths which dictate the fibre deposit sites or paths in the interfacial region 26 will interface functions, meaning single or possibly composite mathematical functions that define particular shapes while still allowing the borders of the regions to continuously transition to each other, rather than simply connecting between the fibre locations in the regions 22, 24. Examples include but are not limited to mathematical functions that form sinusoidal, parabolic, arrowhead and angled shapes. Conceptual illustrations of examples of a sinusoidal interface 1401, an angle interface 1402, and an arrowhead shaped interface 1403 are respectively shown in FIGS. 14-1, 14-2, and 14-3. Example scaffold schematics of layers with sinusoidal shaped, “angled”, and “arrowhead” shaped interfaces transitioning between a region with a rectangular grid pattern and a region where the fibres are arranged in an “auxetic stars” pattern are respectively shown in FIGS. 14-4, 14-5, and 14-6. Parameters used to generate the scaffold schematics of FIGS. 14-4, 14-5, and 14-6 are respectively shown in FIGS. 15-1, 15-2, and 15-3.

The shapes for the interface may be manually defined instead of or in addition to being defined by a function or composite function (i.e., “function defined”). For example, there may be a combination of function or manual definitions for the fibres within one or more of the layers. Interfacial fibres in one or more of the layers may all be manually defined. Interfacial fibres in one or more of the layers may all be function defined.

Further, depending on the fibre arrangement which is to be provided for the regions to be joined by the interface, different print paths intended for different interfacial fibres, each extending between a corresponding pair of fibres from the neighbouring structural regions, can have different shapes or interface functions. In this case, it is preferred that the different interface functions defining a series of adjacent printing paths in the interface region define gradually rather than abruptly changing shapes.

Selection of the interface shapes or functions will depend on various factors, such as the desired printing time and any mechanical properties (e.g., particular bending or flexure requirements) required for the interface region or for the overall tissue scaffold structure. The selection may also depend on values of the MEW process parameters. For instance, a shorter time to traverse through the interface region (and thus a shorter print path) may be required if it is intended that any fusion or bonding occurs between an already deposited fibre portion and a fibre portion to be deposited later (e.g., on a return pass).

The continuous interface for joining two structural regions, further where the interfaces are defined by functions as mentioned above, was demonstrated in experiments conducted by the inventors as providing the greatest degree of flexure compared with conventional techniques for joining two structural. This is useful, particularly for applications such as the heart valve scaffold where flexing or bending in the implanted valve is required to function in a haemodynamic environment, in providing opportunities for fabricating valves which can better maintain their performance over time.

Optionally, two or more of the print paths may be defined such that the deposited fibres along the paths are immediately adjacent each other to form a bundle. The bundles can fuse or bond with each other, under the right MEW settings. This allows the fabrication to provide scaffold portions which emulate anatomical features found in biology. For instance, the inventors observed, in test porcine heart valve samples, splitting of a collagen fibre bundle, and also joining of separate collagen fibres into larger bundles. This is described later in this specification.

In providing the interface via which the two structural regions transition between each other, the pore sizes may be varied between different sections of the printed structure. Three techniques of varying the pore size between sections are proposed: joining, halving and fanning. The same techniques may also be applicable to change the porosity in individual structural regions, instead of or in addition to the transition area (i.e., interface) between different regions.

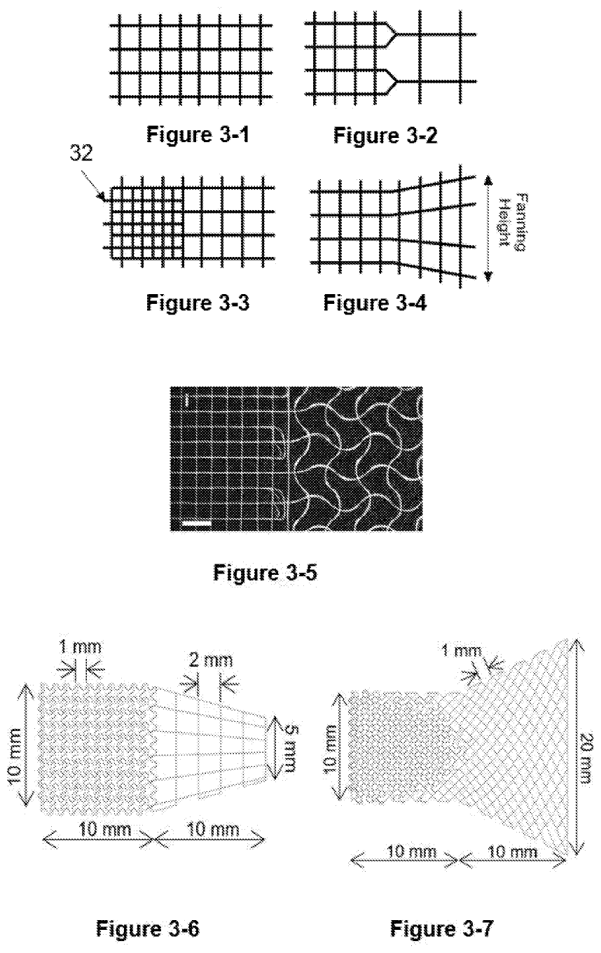

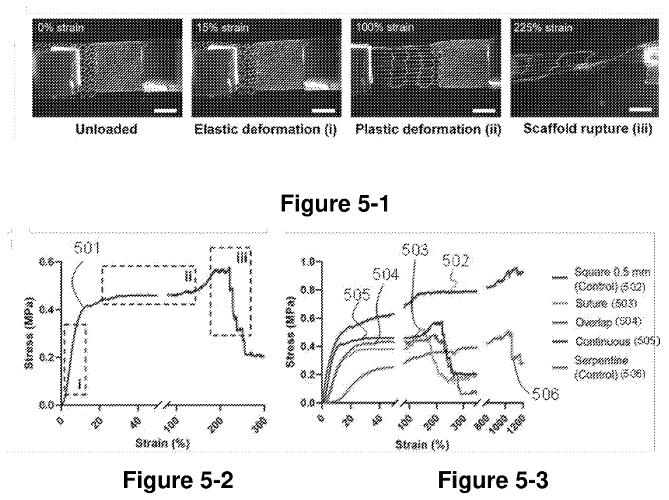

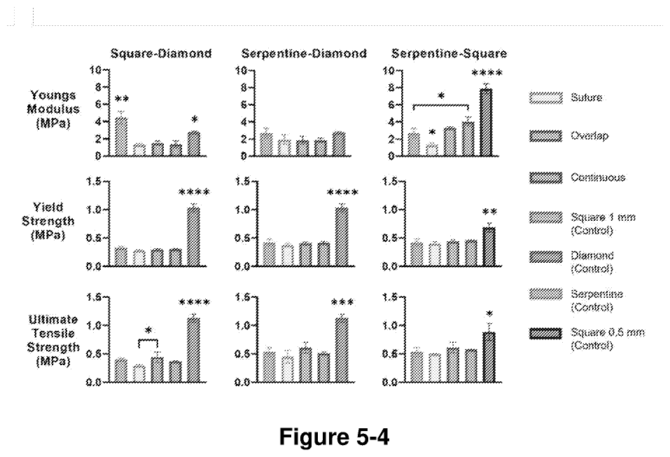

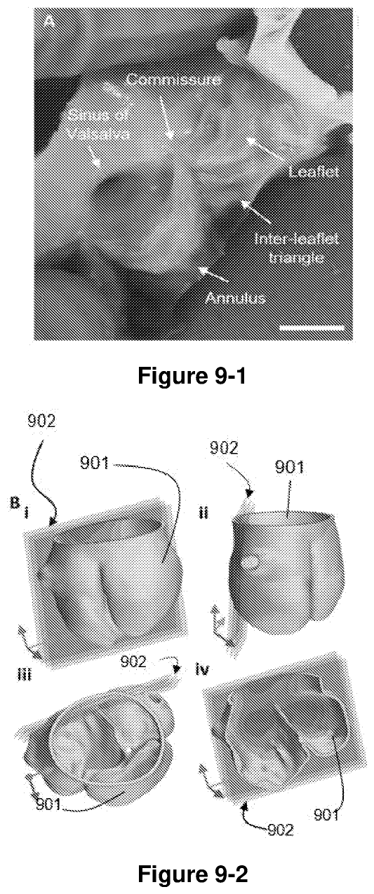

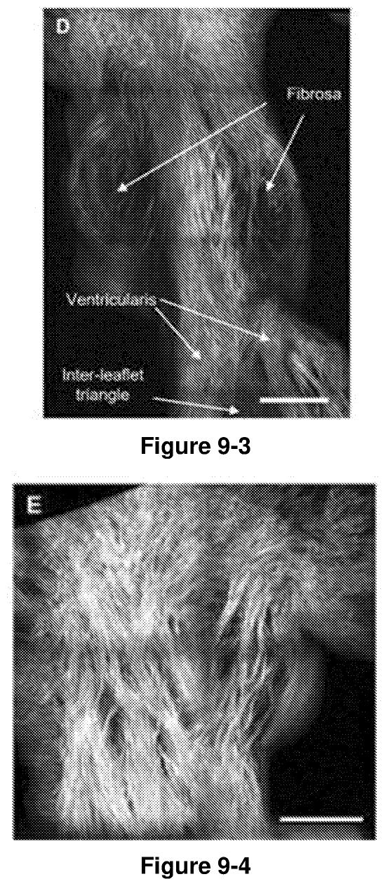

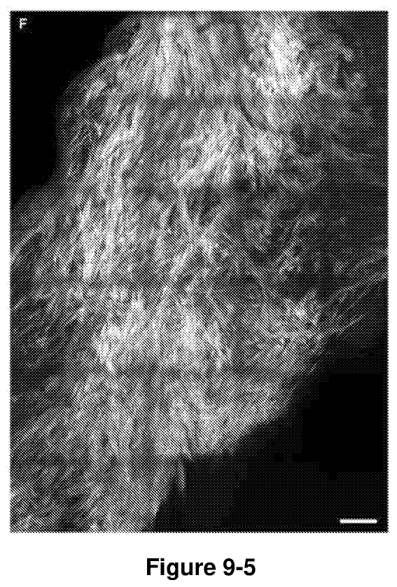

FIGS. 3-1 to 3-4 conceptually depict the “joining”, “halving”, and “fanning” concepts. FIG. 3-1 schematically shows a scaffold with a consistent pore size which is not varied between the left-hand side portion and the right-hand side portion of the scaffold. In the “joining” example of FIG. 3-2, each pair of adjacent fibres from the left-hand side section join together, resulting in a doubling the pore size on the right-hand side. This can be a manner of creating a transition from one structural region to a second structural region which has larger pore sizes. This would also result in the doubling of the number of fibre layers in the second region. This technique echoes the observations made from native tissue (e.g., see FIG. 9-5), where small collagen bundles gradually combine to form thicker bundles, as observed within interfaces elsewhere in the body. The “joining” concept can be generalised so that two or more fibres are joined. Potentially a different number of fibres may be joined in this process at different areas in the scaffold, to create joined bundles of different sizes.

The halving method deposits an additional layer of the scaffold in the first (left hand side) section at an offset equal to half the pore size. This results in a halving of the overall pore size in the first section, while maintaining equal fibre layers between the sections. FIG. 3-3 schematically depicts the halving of the pore size where the additional fibre layer 32 is added. FIG. 3-5 provides an example image of where this occurs in a MEW fabricated device.

The “halving” method can be generalised to decrease the pore size in the region where the additional layer is deposited by an amount which differs than half. For instance, “halving” can be done twice, at a ⅓ pore size offset and a ⅔ pore size offset, to decrease the pore size to ⅓ of the original pore size. Thus, the “halving” concept may be generalised to a “dividing” or “fractioning” concept.

The fanning method causes a gradient change in porosity. The gradient change can be set to occur over a “fanning height” which is pre-defined or selected by the designer of the MEW scaffold. Fanning can occur to gradually decrease the pore size (FIG. 3-4, FIG. 3-6) or to gradually increase the pore size (FIG. 3-7).

### EXPERIMENTS

### Example 1

Comparison of Continuous Interfacing with Other Methods of Interfacing Multi-Phasic MEW Scaffolds

The effect of the design of the interfacial region on the behaviour of the printed scaffold was systematically investigated. Aside from the continuous interfacing described above, two other interfacing methods, including overlapping and suturing, were also investigated. See FIG. 4-1 which includes schematic depictions of a continuous interface, a suturing interface, and an overlapping interface.

Three different patterns were chosen (squares, diamonds, and serpentines) to be combined using the three interfacing printing strategies to produce bi-phasic MEW scaffolds. Scaffolds were fabricated from medical grade poly(ε-caprolactone) (PCL) to make 5-layered, 10 mm by 40 mm scaffolds, comprising two 20 mm patterns and an interfacial region. The size of the interfacial region depended on the interfacing method used: overlaps were 1 mm, sutures were 2 mm, and the continuous method resulted in a gradual, undefinable transition region. To demonstrate the capability of the three methods to interface spatially heterogeneous scaffolds, the pore size for each pattern was varied between 1 mm and 0.5 mm. The square pattern was chosen to alternate pore size, while the diamond and serpentine pore size were kept constant at 0.5 mm and 1 mm, respectively. Pore sizes were chosen to facilitate ease of printing and delineation of the effect of interfacing method independent of the scaffold's applicability to tissue engineering. Control scaffolds (40 mm by 10 mm single pattern scaffolds with no interface) were also printed to test each pattern and pore size independently of any interface. Average fibre diameter was 26.09±1.90 μm. FIG. 4-2 shows exemplary images of each of the interface types for the square-to-diamond scaffolds.

Next, how well each interfacing method resembled the programmed print path was assessed by comparing their morphologies using light microscopy and scanning electron microscopy (SEM). Bridging of fibres, a known imperfection where fibres will deviate from their intended path onto an adjacent fibre due to electrostatic repulsion caused by residual charge, was identified in varying quantities across all scaffolds. Overlapping scaffolds exhibited the densest interfacial region, with some cases of bridging. The suturing method was more customisable, but for the chosen design presented slightly less fibre density and comparable amounts of bridging to the overlapping scaffold. The continuous printing technique best matched the planned print path with fewer fibre bridging defects and the lowest density. These results were consistent with literature, as the amount of printing defects in MEW scaffolds are expected to increase in areas of higher fibre density as well as in areas where the jet changes direction abruptly. Thus, continuous interfaces produced more accurate prints and exhibited a gradual transition of densities between spatially heterogeneous regions, as opposed to a dense band containing more fibres.

### Example 2

### Effect of Interfacial Printing Methods on the Mechanical Properties of Bi-Phasic MEW Scaffolds

### Uni-Axial Tensile Testing

To assess the effect of the interfacial printing method on the mechanical properties, uni-axial tensile testing was performed on the bi-phasic MEW scaffolds with loading perpendicular to the interface. FIG. 5-1 includes time lapsed images take at different stages of the stretching, being from left to right, the unloaded stage, elastic deformation stage, plastic deformation stage, and scaffold rupture. FIG. 5-2 depicts a representative stress-strain plot of continuously interfaced scaffold with labels corresponding to regions of i) elastic deformation, ii) plastic deformation, and iii) scaffold rupture. FIG. 5-3 depicts combined representative stress-strain plots for the serpentine-square scaffolds with each type of interface and control scaffolds. FIG. 5-4 depicts Young's modulus, yield strength and ultimate tensile strength for each type of bi-phasic scaffold with interface method and control scaffolds (average over n=3, error bars represent one standard deviation, significant differences were assessed using one-way ANOVA with Tukey's multiple comparisons test, where statistical significances are shown over a singular column this data had at least that level of significant difference when compared to every other column on the graph, *p≤0.05, **p≤0.01, ***p≤0.001, ****p≤0.0001).

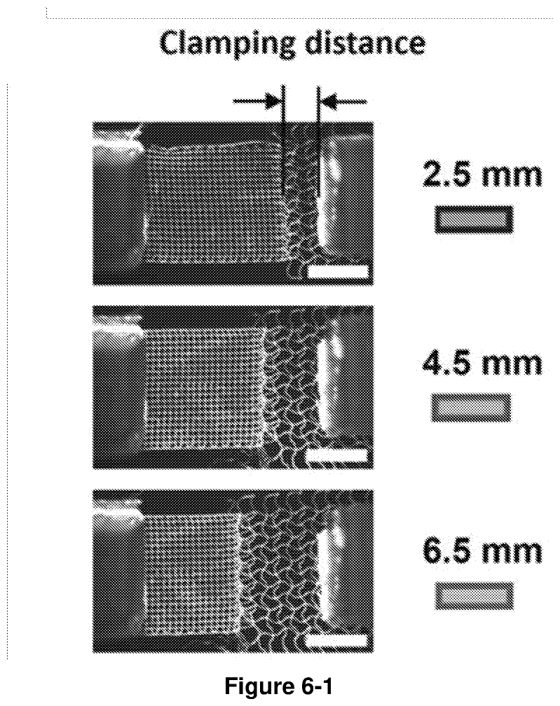

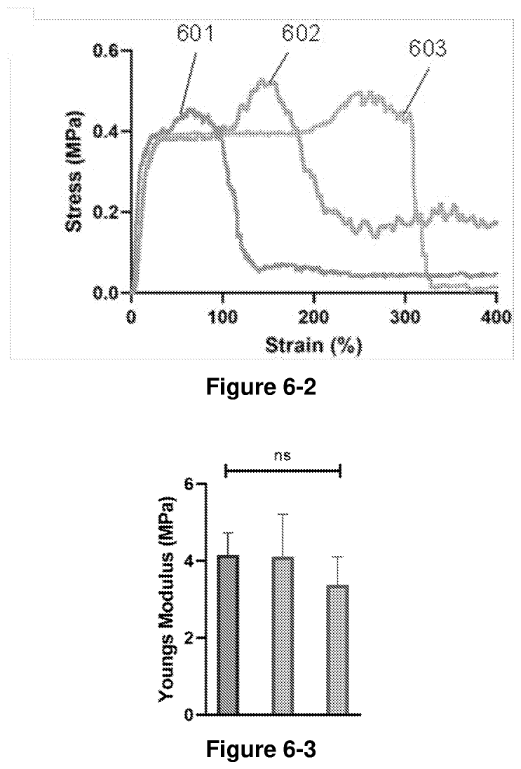

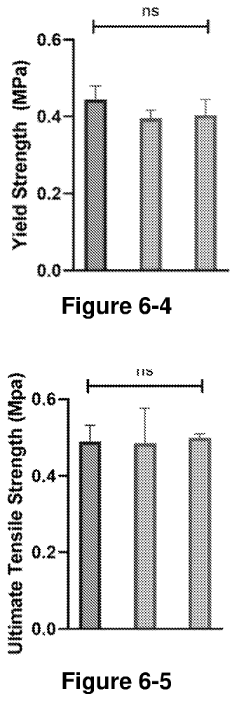

FIG. 6-1 depicts images of a continuously interfaced square-to-serpentine scaffold to which uni-axial straining forces were applied at different clamping distances. The effect of clamping distance from the interface on mechanical results was first investigated and no significant difference was observed in the calculated stresses. Stress parameters including Young's Modulus, yield strength, and ultimate tensile strength are depicted in FIGS. 6-3, 6-4, and 6-5. In each of FIGS. 6-3, 6-4, and 6-5, the results depicted in the order from left to right correspond with results obtained with clamping distances of 2.5 mm, 4.5 mm, 6.5 mm, respectively.

However, strain values were observed to vary significantly and thus related metrics such as yield strain cannot be compared. Subsequently, due to the limited strain testing range of the equipment, scaffolds were clamped closer to the interface on the side of the weaker scaffold. FIG. 5-2 shows a representative stress-strain plot 501 of the continuously interfaced serpentine-to-square scaffold. The initial elastic deformation (i) followed by plastic deformation (ii) of the scaffolds, was accounted for almost entirely by the weaker pattern, in this case the serpentine. The fibres of the weaker scaffold then began to neck resulting in strain hardening, shown by a gradual increase in stress. When the scaffold reached its ultimate tensile strength (UTS), the scaffold ruptured (iii) indicated by the sharp decrease in stress corresponding to the strain at which the two patterns separated from each other. A similar stress-strain curve was common among the rest of the interfaced scaffolds, as shown in FIG. 5-3. Stress-strain plots and scaffold testing for the serpentine-square, square-diamond and serpentine-diamond scaffolds showed similar behaviour.

The Young's modulus of the interfaced scaffolds was dominated by the more elastic, meaning lower modulus, of the two interfaced patterns (FIG. 5-4). In all except one case (continuous serpentine-square) the elasticity of the interfaced scaffold was either equal to or greater than that of the more elastic pattern. For both the square-diamond and the serpentine-diamond scaffolds, the Young's modulus was not affected by the interfacing method. Interestingly, interfacing the square-diamond scaffolds resulted in a significantly more elastic scaffold than either of its constituents, irrespective of interfacing method. In the serpentine-square scaffolds however, the suturing method was the only technique resulting in a more elastic scaffold. These results suggest that despite the majority of the elastic behaviour coming from the more elastic pattern, both patterns contribute in some manner to the final scaffolds elasticity. The yield strength indicates the level of stress at which the scaffolds will start to deform permanently. Results showed that yielding was dictated entirely by the weaker pattern, meaning the pattern with lower yield strength. Similarly, the UTS of the scaffolds was primarily determined by the weaker of the two patterns bar one case (suture square-diamond). Overall, the interfacing method did not have a significant effect on uni-axial mechanical properties due to the dominance of the weaker scaffold. However, other mechanical properties relevant to tissue-engineered scaffolds may be impacted.

### Flexural Testing

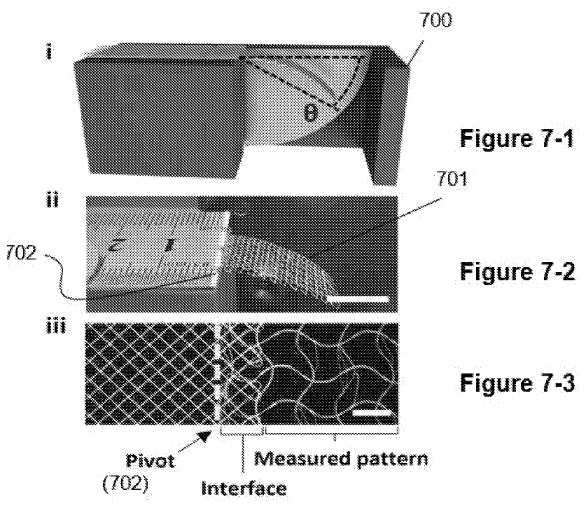

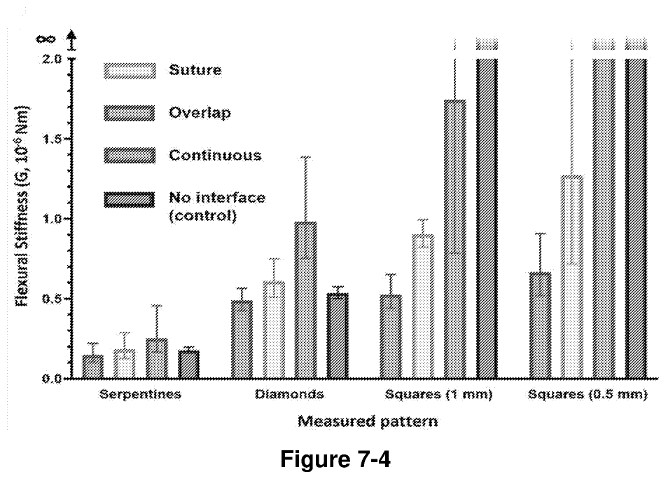

Next, how the printing method used to interface two different patterns would alter the flexural properties of the resultant bi-phasic scaffolds was investigated. To do so, a flexural testing apparatus 700 schematically represented in FIG. 7-1, in accordance with ASTM D1388, was built. FIG. 7-2 is a close-up view of a bi-phasic scaffold 701, with an imaginary dashed line 702 representing the location of the pivot line. Scale bar=1 mm. FIG. 7-2 shows an image of testing of multi-phasic scaffold with serpentine pattern. Flexural stiffness data are shown in FIG. 7-4, (averaged over n=3, error bars represent standard deviation, two-way ANOVA with Tukey's multiple comparisons test resulted in p<0.0001 for effect of pattern, p=0.0002 for effect of interface and non-significant differences between individual means).

During test, each of the scaffolds was pivoted immediately on one side of the interface, leaving one pattern fixed, and one flexing, from which the bending angle was measured, and subsequent flexural stiffness (G, measured in Nm) calculated. G values for the control scaffolds were also calculated. Lower G values indicate a more flexible scaffold. Flexural properties depended largely on the pattern of the flexing scaffold, as can be seen from FIG. 7-4. Amongst the patterns tested, serpentine scaffolds were the most flexible (least flexural stiffness), followed by diamonds, 1 mm squares, and 0.5 mm squares. Note that, as the bending angle, θ, trends to 0°, G trends to infinity, meaning very stiff scaffolds that barely flex had exponentially higher values for G. This was observed for both the square patterns, which have distinguishably higher values for G. Interestingly, the type of interface significantly influenced flexural stiffness. In particular, for stiffer patterns, both suture and continuous interfaces enabled greater flexure for the stiffer square patterns, with continuous interfaces enabling the greatest increase in flexure. Contrastingly, overlapping interfaces often had a detrimental effect on flexure. For already flexible patterns, such as the serpentine and diamond, the interface had a negligible effect. Comparing interface techniques overall, a correlation can be observed whereby continuous interfaces offer the greatest degree of flexure, followed by suture and then overlap. Interestingly, a similar trend was observed earlier when visually analysing fibre density in the interfacial region between methods. Hence, this may be a possible mechanism behind the observed flexural properties, whereby an interface with higher density and more sites of fibre fusion will result in greater flexural stiffness.

In summary, from the test results, interfacing MEW scaffolds will, in most cases, not weaken the scaffold's mechanical performance and often result in equal or greater elasticity than the individual constituents. Furthermore, using a continuous interfacing approach is recommended when trying to achieve greater flexural properties. This is valuable within the context of interfacial tissue engineering, as specific flexural properties may be desired across the interface between two regions. In the context of a tissue-engineered heart valve scaffold, a high degree of flexure is required for the interface between the leaflet and inter-leaflet triangle, as large amounts of cyclic bending will occur. This strategy could therefore continue to be leveraged to further unlock the capabilities of MEW scaffolds.

### Example 3

User-Generated MEW Scaffolds with Complex Continuous Interfaces

Writing G-code for continuous printing of MEW scaffolds can be time consuming, especially in the case of complex scaffold architectures with multiple regions. A graphical user interface (GUI) can be written to utilise GUI technology and computing advances to more rapidly generate MEW scaffolds which includes tailorable interfaces using continuous printing.

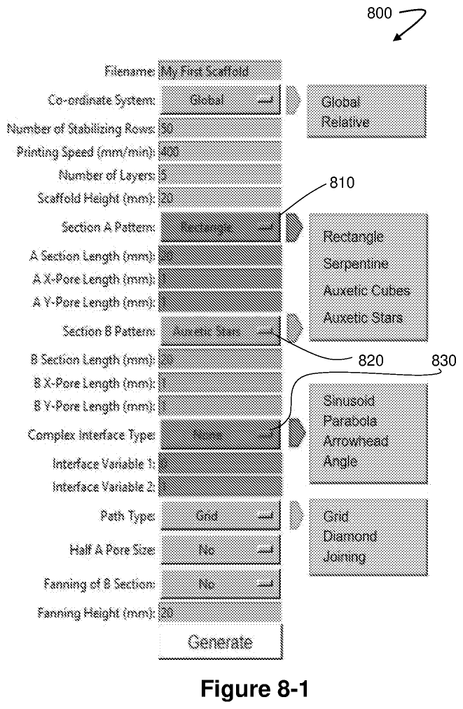

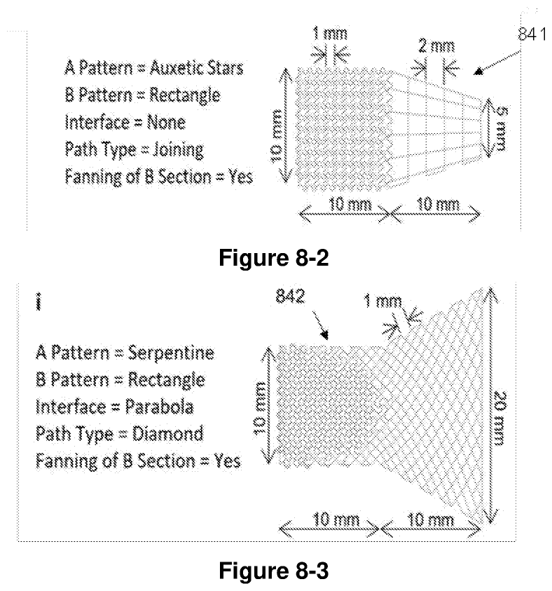

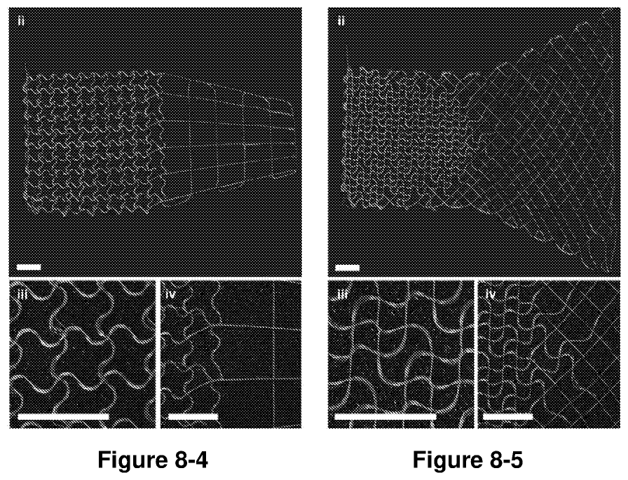

A screen shot of the GUI 800 is shown in FIG. 8-1, for designing a scaffold having an interface between two sections “A” and “B”. The inventors previously proposed novel MEW scaffold structures where each of the regions comprises intersecting sets of fibres, where each set includes fibres which are in parallel or approximately parallel alignment with each other. This results in a pore shape which is the same or about the same within each section.

For simplicity, the GUI 800 is provided for designing a scaffold of the above-mentioned type. The scaffold further has a continuous interface as discussed in this document. The GUI 800 provides selectable fields 810, 820 where the patterns (i.e., shape) of the pores for each section can be chosen. Rectangles, serpentines, auxetic cubes, and auxetic stars are included as examples and are not intended to be limiting. Further fields for the selection of section lengths, pore sizes and path types are also included in the GUI 800. In this example, the “path type” choices are “grid” where the fibres are laid in horizontal and vertical lines forming a rectangular grid, “diamond” where the fibres are laid in diagonal lines, and “joining” where joining of two or more fibres occur. It will be appreciated that other path types may be defined and presented as options. At the GUI field 830 the interface function for the continuous interface can be further set to be a mathematical function by choosing one of the function choices, allowing the border between sections “A” and “B” to be dictated by the mathematical function while still being continuous. Non-limiting example functions here include sinusoidal, parabolic, arrowhead and angled shapes. GUI field 840 also allows the selection of how to vary the Y-pore size between sections, including halving and fanning, as described above in relation to FIG. 3.

FIGS. 8-2 and FIG. 8-3 respectively show two exemplary MEW scaffolds designs and the input parameters used to in the designs In FIG. 8-1, the scaffold 841 is designed to employ joining and fanning methods. In FIG. 8-2, the scaffold 842 is designed to employ diagonally directed paths (i.e., the path type is selected to be “diamond”) where fanning occurs in one section, Digital microscopy images of MEW scaffolds fabricated using PCL are shown in FIGS. 8-4 and 8-5.

The inventive method has the capabilities to generate continuous, user-defined MEW scaffolds. This novel method and its implementation using e.g., GUI unlock the ability for rapid, iterative design of a wide range of continuously interfaced scaffolds, offering a wide range of tunability of mechanical and morphological properties that could be highly beneficial to interfacial tissue engineering applications using MEW.

### Example 4

### Morphological Analysis of the Aortic Heart Valve Interfaces

In experiments conducted, multimodal imaging was applied to examine the morphology of the interfacial regions of the aortic heart valve as the basis to establish a functional bio-inspired design for MEW scaffold interfaces. Subsequently, three methodologies for interfacing heterogeneous scaffolds were investigated to understand their effect on tensile and flexural properties. Combining the native tissue investigation and study on interfacing methods, an integrated biomimetic heart valve interface was designed, fabricated and tested.

The aortic valve leaflets are responsible for the majority of the valve's overall functionality and have been the focus of previous studies. Moreover, other regions of the aortic valve are also known to significantly contribute to the valve's functionality. These regions have been classified in differing ways. For the purpose of this specification, the regions are divided into leaflets, sinuses of Valsalva, annulus, commissures and inter-leaflet triangles as shown in FIG. 9-1. Each region has different concentrations and orientations of extracellular matrix (ECM) constituents, including collagen and elastin fibres which are of particular interest when considering the mechanical functionality of the valve. Collagen fibres are the main load bearing component of the ECM and are responsible for the anisotropic mechanical characteristics and J-shaped stress-strain response of the tissue. Elastin fibres play a role in the low-strain performance of the valve but more importantly regulate the collagen fibre orientation, ensuring they return to their pre-loaded state between cycles. Hence, while all components of the ECM play an important role in the valve functionality, our investigation focusses on the orientation of collagen fibres to inspire MEW scaffold design. It is thus important to understand what is already known regarding collagen fibre orientation in the heterogeneous regions of the aortic valve, which is outlined briefly below.

The aortic valve leaflets contain three distinct layers: the fibrosa, spongiosa and ventricularis (from aorta to ventricle). The tensile load bearing layers are the fibrosa and ventricularis, which consist mostly of circumferentially and radially aligned collagen fibres, respectively. The sinuses of Valsalva are similar in composition to the aortic wall, consisting of three distinctive layers where the inner (intima) and outer (adventitia) comprise of primarily longitudinally arranged collagen fibres, whereas the middle (media) layer is predominantly circumferentially arranged fibres. The annulus is a fibrous structure connecting the leaflets and sinus wall into the left ventricle, and consists mainly of highly dense, circumferentially aligned, collagen bundles, resulting in relatively rigid mechanical properties. The commissure exists at the point where the free edge of the two leaflets meet. By helping transmit forces between the leaflets and the surrounding aortic root, the commissure plays a crucial function in supporting both the flexural properties required to open the valve in diastole as well as withstanding high tensile loads as the valve closes in systole [66]. With respect to the microstructure, collagen fibres primarily extend radially from the leaflets, intertwining through the commissural region and anchoring into the aortic wall, enabling transmission of forces between the leaflet and root. The inter-leaflet triangles are governed by the region below the commissure, between the leaflets, and above the annulus. The orientation of the fibrous microstructure is largely unexplored in the inter-leaflet triangle; however, it is thought to primarily follow that of the annulus, that is circumferentially. Notably, while the collagenous microstructure of these regions is known, the specific nature of how the collagen fibres orientate in the interfaces between these regions remains to be investigated. This could provide valuable insight to inform the design of scaffolds striving to mimic native fibrous morphology. Thus, the inventors began the problem solving process by investigating whether high resolution multi-modal imaging of porcine tissue could provide insight into some of these unknowns.

Experimentation and analysis using porcine tissue has been adopted widely in cardiovascular research owing to the greater availability of tissues with anatomical and haemodynamic similarities to humans. When analysing the collagenous microstructure in these studies, the regional fibrous orientation was consistent between human and porcine species. Accordingly, porcine tissue has been used for biomechanical studies, and to elucidate fibre alignment using microscopy.

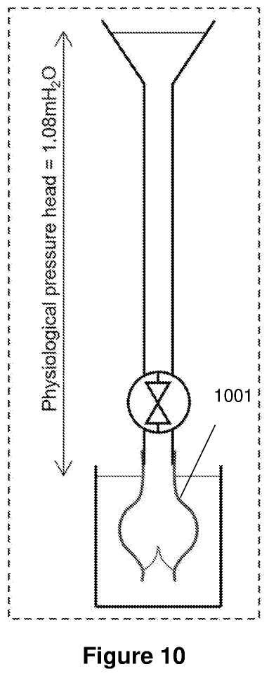

As schematically depicted in FIG. 10, porcine aortic valve tissue 1001 was fixed using a hybrid immersion fixation and hydrostatic pressure distension method to preserve near-physiological diastolic conditions. First, microcomputed tomography (micro-CT) was used for the 3D reconstruction of the valve architecture, to facilitate macroscale identification of regions of interest. Next, tissue was sectioned longitudinally through the wall of the aortic root, parallel to the direction of blood flow, at a thickness of 250 μm for further analysis via second harmonic generation (SHG) imaging. FIG. 9-2 depicts the 3D reconstructed valve architecture 901 and the sectioning planes 902, as viewed from the i) coronal (external), ii) sagittal, iii) axial, and iv) coronal (internal) directions. This sectioning orientation allowed for imaging of the commissure and inter-leaflet triangle, the interface between them, as well as the interfaces with the leaflets, sinuses, and annulus. Collection of three slices into the aortic wall enabled for confirmation of orientation information in the third dimension. SHG is an optical microscopy technique ideally suited for imaging collagen from the tissue scale to molecular scale due to the second order non-linear characteristic of collagen fibres.

The microstructure of specific heart valve regions was identified in SHG images (e.g. FIG. 9-3), including the commissure, and the fibrosa and ventricularis layers of two adjacent leaflets. In the commissure, large amounts of collagen fibres were densely interlaced and intertwined between the vertical and horizontal directions. Three distinct groups of collagen fibres travelled down from the top of the commissure into the leaflets. The left and right of these groups merged into the fibrosa layers of the two adjacent leaflets, which can be identified by their distinctly rounded structures with collagen fibres travelling into and out of the page, showed by a diminishing SHG signal. Interestingly, we observed that the centre group of fibres travelled down from the top of the commissure for approximately 500 μm before splitting into the ventricularis layer of each leaflet, identifiable by the vertically aligned collagen. To our knowledge, the orientation and extent to which the commissure interfaces with the fibrosa and ventricularis layers of two adjacent leaflets was unknown until now. In a deeper image of the commissure, we observed highly aligned collagen fibres travelling in the horizontal direction from the sinus, before joining into larger bundles as they turned to travel vertically downwards, by which point the fibres were strongly aligned and closely bundled again (FIG. 9-4).

SHG imaging of the inter-leaflet triangle revealed horizontally orientated bundles of collagen fibres that emerged from the leaflet and sinuses on either side (see FIG. 9-5). Fibres regularly intertwined as they transitioned from horizontal, to diagonal and then primarily vertical orientation. The vertically aligned bundles exiting the top of the image continue into the commissural region. Changes in fibre direction were gradual and accompanied by fibre recruitment into discretised bundles as they pivoted before reaching vertical or diagonal alignment.

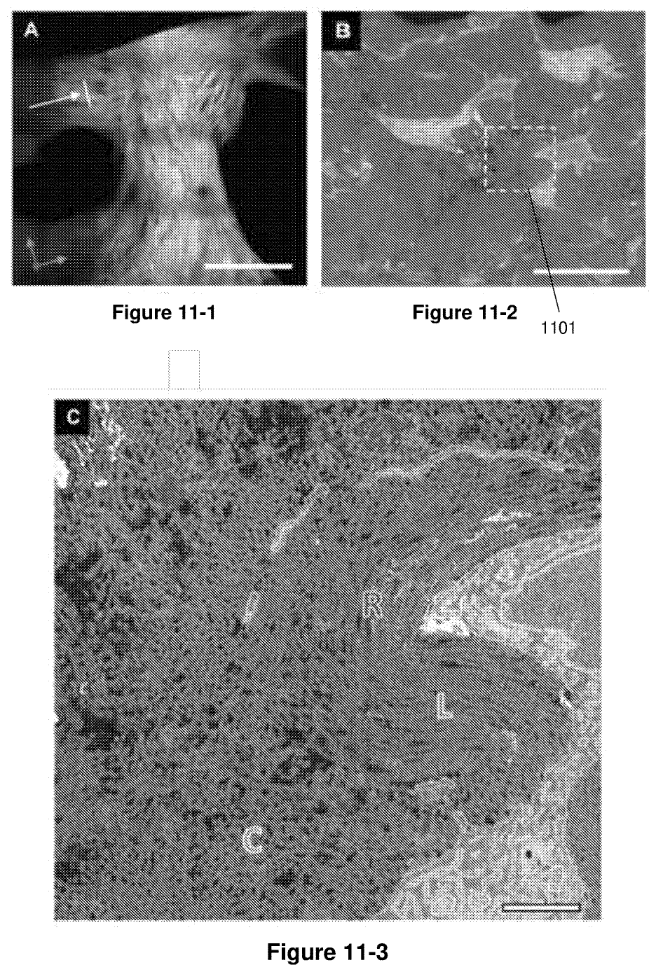

In order to validate SHG collagen fibre orientation data, correlative focused ion beam scanning electron microscopy (FIBSEM) imaging was conducted on the same samples. A specific region from the SHG images was selected and co-registered to the FIBSEM slice (FIG. 11-1). At low magnification, the FIBSEM slice shows collagen fibre bundles filling the space between the valve fibrocytes (FIG. 11-2). FIG. 11-3 is a higher magnification image of the boxed area 1101 shown in FIG. 11-2. At higher magnification, individual collagen fibre cross sections can be seen grouped together in bundles, travelling primarily circumferentially C, as well as one bundle changing orientation from radial to longitudinal (R, L). This agrees with observations from SHG imaging in FIG. 11-1, where collagen fibres primarily travelled circumferentially, perpendicular to the plane of the slice, whilst gradually turning to run longitudinally, parallel to the same plane as the slice.

From the multi-modal imaging analysis of the porcine aortic valve, we can summarise the following: 1) The commissure consists of a complex interwoven network of collagen fibres from multiple directions, which then coalesce into a primarily vertical alignment at the core of the commissure; 2) in the inter-leaflet triangle region, collagen fibre bundles exhibit a regular diagonal weave before coalescing towards primarily vertical alignment, bundling more as they transition into the commissure; and 3) fibre orientation changes across interfaces were gradual, and often characterised by a transition from uniformly aligned large fibrous sheets, into more discrete bundling as fibres turn at more acute angles. Due to the complexities associated with developing a sample preparation method compatible with precise correlative imaging across modes, and the large time taken to execute this, a limitation of this study was n=1 samples. The observations from this high-resolution multi-modal imaging analysis performed on the aortic heart valve interfaces were used in this study as inspiration for interfacing fibrous heart valve scaffold designs.

### Tissue Sourcing and Preparation

Collection and use of porcine animal tissue for this work was approved by the University of Western Australia Institutional Biosafety Committee (F 69199). Porcine hearts were sourced from the University of Western Australia Large Animal Facility, where tissue was excised within 2 hours of euthanasia. Immediately after removal, hearts were dissected to leave the aortic roots with their valves, sinuses, the first few millimeters of the coronary arteries and a portion of the ascending aorta.

Primary fixation of (n=1) tissue was in fresh 4% paraformaldehyde solution (Cat #C007, ProSciTech, Australia) made in 0.1 molar phosphate buffer (PB) (pH 7.4). Fixation was conducted for 1 hour at room temperature and equivalent to diastolic pressure (80 mmHg) with the hybrid immersion fixation and hydrostatic pressure distension apparatus shown in Figure S1 (Supporting Information). Secondary fixation was via immersion overnight in 2.5% glutaraldehyde (Cat #EMS16400; ProSciTech, Australia) in 0.1 molar PB (pH 7.4). Excess tissue was removed by dissection before storage in glutaraldehyde fixative solution at 4° C.

### Micro-Computed Tomography

During imaging, tissue samples were kept moist under paper towel soaked with glutaraldehyde solution and placed inside a sealed zip lock bag. Micro-CT imaging (Skyscan 1176, Bruker-microCT, Kontich, Belgium) was performed at a source voltage of 45 kV, source current of 556 μA, 86 ms exposure time, 34.81 μm/pixel resolution, with a 0.2 mm aluminum filter, 0.7° over 360° rotation with 2/image frame averaging enabled. The micro-CT data was segmented and reconstructed using in-house software before being brought into STAR-CCM+ (v16.04.012-R8, Siemens) for further smoothing and processing.

### Second Harmonic Generation Imaging

A 5% agarose (Agarose LE, analytical grade, Promega, Australia) embedding solution was used at 65° C. Tissue samples were removed from the fixative solution, and blotted dry, before being placed into a custom sized (approximately 30 mm cubed) Lego (The Lego Group, Denmark) mould that contained the entire volume of the tissue and minimised the amount of agarose solution used. The agarose solution was decanted around the tissue, ensuring minimal bubbles remained. Embedded tissue was cooled to room temperature, then stored at 4° C. overnight. For sectioning, the solid gel/tissue block was removed from the mould and superglued to the vibratome stage (Vibratome 3000, The Vibratome Company, St Louis, MO, USA). After sectioning the block face to the region of interest, three consecutive sections were taken at 250 μm thickness. Optical imaging was then conducted on these slices using an inverted A1RMP multi-photon microscope (Nikon) equipped with 10×/0.45NA objective lens (Nikon) and tuneable laser (10 mW output; 900 nm emission wavelength). All images were captured using NIS-Elements AR software (v5.30.02; Nikon).

### Electron Microscopy of Tissue

Electron microscopy samples were prepared from sections after SHG imaging by microwave assisted processing, using a BioWave Pro microwave system (Pelco). Briefly, the samples were osmicated using the R-OTO method, en bloc stained with aqueous uranyl acetate & lead aspartate, then dehydrated through graduated ethanol series (80%, 90%, 95%, 100%, 100% (v/v)) and propylene oxide (100%, 100% (v/v)). Sample infiltration with Araldite 502/Embed 812 was performed via graduated concentration series in propylene oxide under vacuum (25%, 50% 75% 100%, 100% (v/v)). The samples were polymerized at 60° C. for 48 hours and trimmed on an ultramicrotome (Leica UC6, Leica Biosystems) in preparation for imaging on a FEI Helios Nanolab G3 CX DualBeam FIB-SEM (Thermo Fischer Scientific). The target region was milled using a Gallium FIB current of 65 nA at 30 kV, before backscatter electron imaging of the block-face at an accelerating voltage of 2 kV in magnetic immersion mode.

### Example 5

### Design, Fabrication and Testing of a Bio-Inspired Aortic Heart Valve Interfacial Scaffold

Leveraging the novel microstructural information of the aortic valve interfaces gained from the multi-modal imaging investigation, a bio-inspired MEW scaffold of the aortic heart valve interfacial region was designed using complex continuous interfaces. First, orientation analysis of the collagen structures from FIG. 9-5 enabled a quantitative understanding of the micro fibrous collagen orientation. For example, see FIGS. 12-1, 12-2, and 12-3, which show a colour-mapped orientation analysis of the SHG images of the inter-leaflet triangle region (each scale bar=0.5 mm). In the tissue sample, gradual changes in fibre distributions were observed from 0° (horizontal), to peaks at ±50° (diagonal), and then gradual changes again to ±90° (vertical) fibre alignment. Interestingly, few fibres were entirely vertically orientated, with the majority being woven in a regular diagonal pattern with a slight vertical inclination.

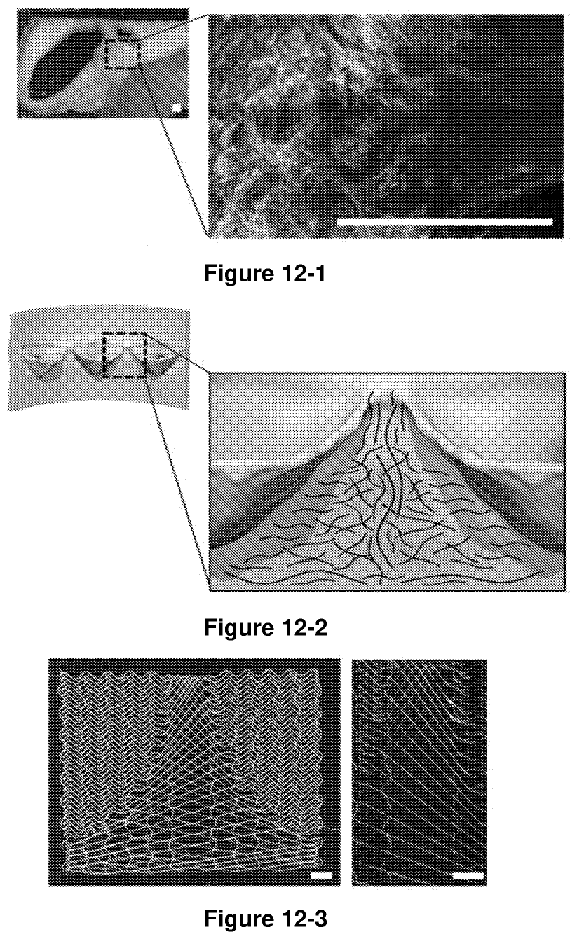

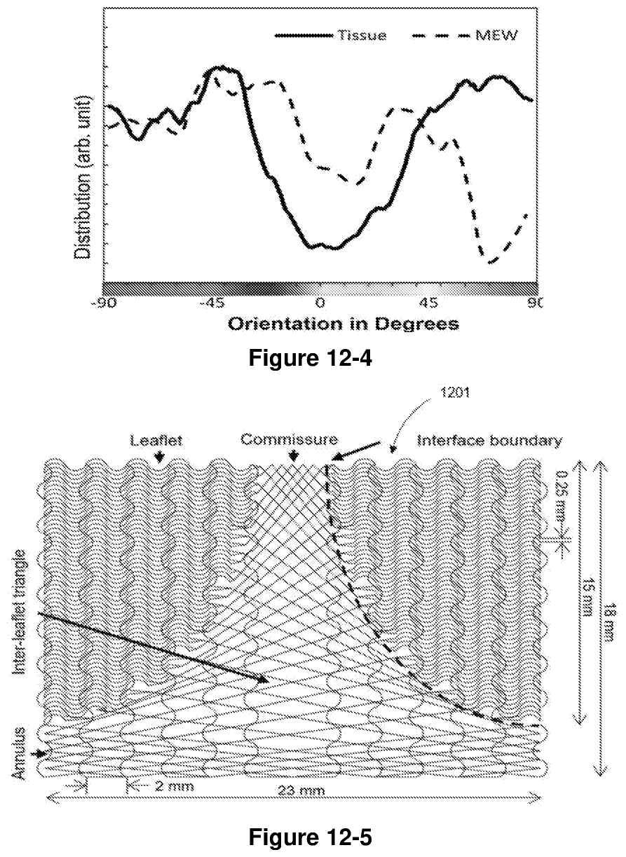

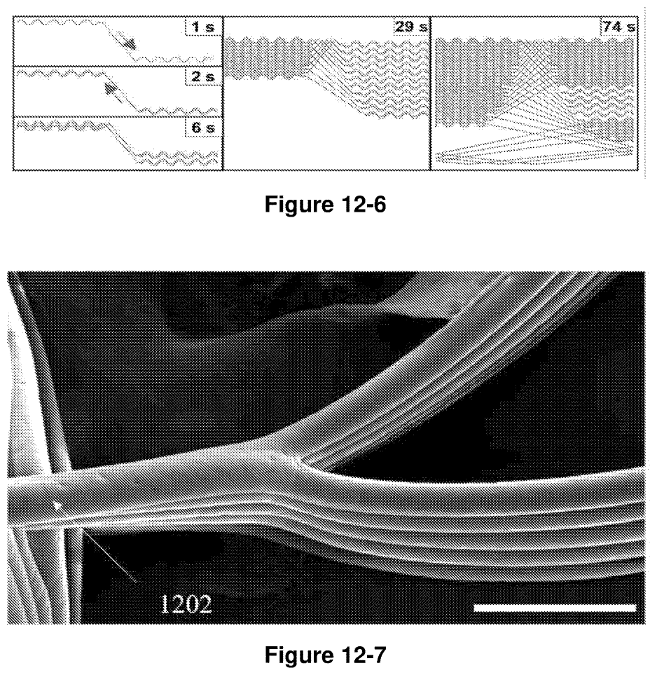

The intricate and complex woven fibrillar structure of the inter-leaflet triangle region was simplified into schematic form (FIG. 12-2) to facilitate scaffold design. The pattern in the leaflet region was based on the inventors' previously published heart valve leaflet design (Saidy et al (2019) Small 15 (24), https://doi.org/10.1002/smll.201900873) For the region 1201 between the leaflets, as opposed to being discretised into commissure, inter-leaflet triangle, and annulus, a gradient diamond pattern was utilised, as can be seen in FIG. 12-3. The gradient transitions from primarily horizontal alignment at the bottom, which aimed to emulate the circumferential fibres present in the annulus, to a primarily vertical alignment at the top, mimicking the longitudinal fibres present at the commissure (visible in FIGS. 9-3, 9-4).

The fibre design avoided entirely horizontal or vertical orientation, as straight MEW fibre patterns are more rigid and weaker than diamond patterns of the same porosity, as could be seen from the data shown in FIG. 5-4. Similar distribution of fibre orientations was achieved when comparing the fibres in the MEW scaffold design with those previously analysed in native tissue, as shown in FIG. 12-4. Hence, the resulting scaffold design combined the native tissue observations with continuous interfaces to create a biomimetic scaffold design with gradient porosities, region-specific layer numbers, tailored fibre orientations and spatially heterogeneous regions.

The above-mentioned process for designing an MEW heart valve scaffold can be generalised for designing MEW scaffolds for other soft tissues.

Scaffolds 1201 of the configuration shown in FIG. 12-5 were successfully fabricated from PCL with an average fibre diameter of 27.69±4.66 μm. FIG. 12-6 shows snap shots of the deposited fibre, taken at times at 1 second, 2 seconds, 6 seconds, 29 seconds, and 74 seconds after the start of the MEW printing, to illustrate the continuous printing path.

The continuous printing path was designed to employ the joining technique employed transition continuously from 5 layers in the leaflet region to 10 layers in the rest of the scaffold. In some cases, where a double print layer was deposited due to the joining method, fibre fusion occurred simultaneously, resulting in thicker fibres instead of two layers of fibres. The fused, thicker fibre 1202 can be seen from FIG. 12-7. The average fibre diameters for non-fused and fused fibres were 24.34±0.75 μm and 33.27±2.30 μm, respectively. Comparison of the cross-sectional areas of fused to non-fused fibres confirmed fibre fusion phenomena with fused fibres having approximately double (187±14%) the area. This was likely due to the high temperature of the deposited fibre being maintained caused by the very short time between fibre deposition on the same path. This was reminiscent of the morphological arrangement of collagen fibrils found in native tissue, where smaller collagen bundles join together to form larger, tightly packed bundles as they change orientation.

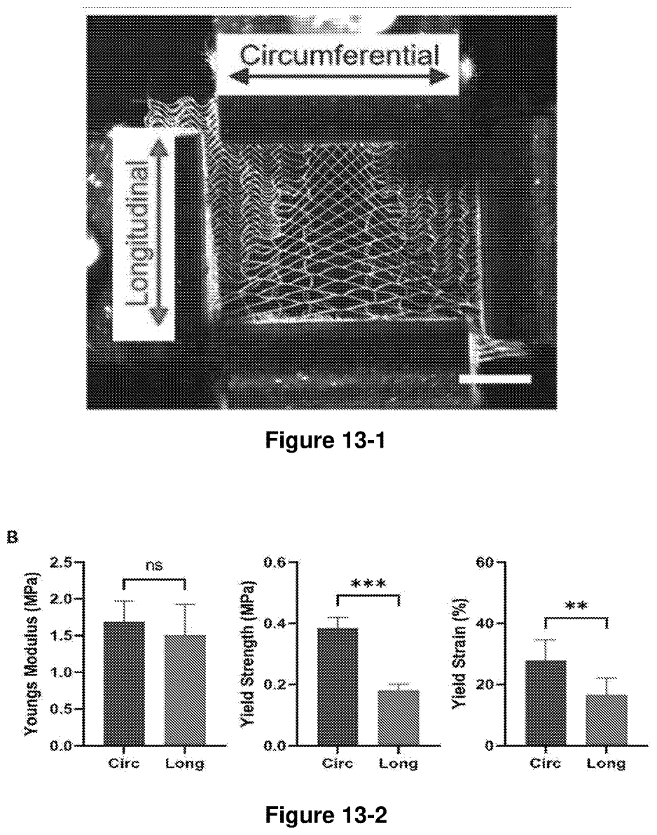

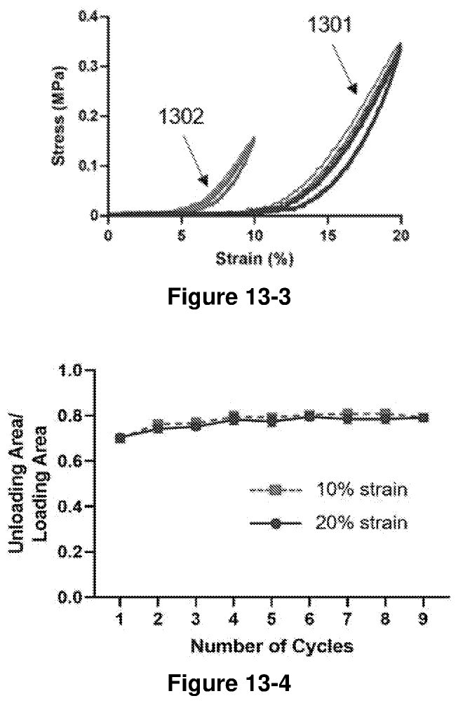

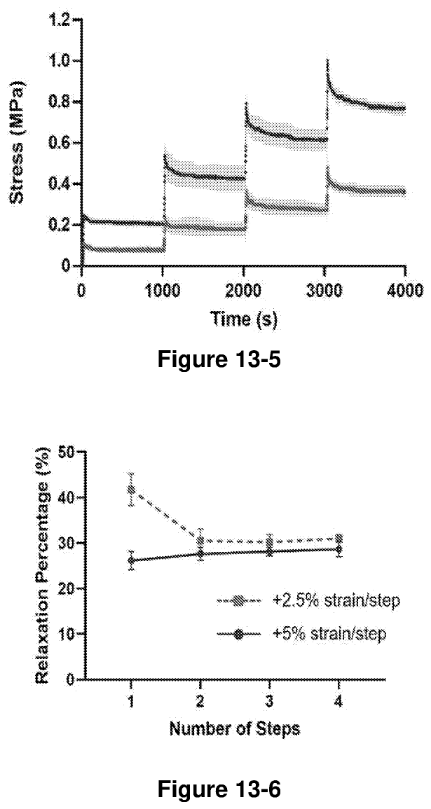

The scaffolds were then mechanically characterised under bi-axial, physiologically relevant strains in the “circumferential” direction and in the longitudinal direction as depicted in FIG. 13-1. Relative strain-related metrics were used to understand tissue-like characteristics such as anisotropy and viscoelasticity were studied. Furthermore, it is important to note that this scaffold contained both the leaflet, interleaflet triangle, annulus, and commissural regions, meaning that comparisons to region-specific data were not strictly physiologically accurate. Attempts to assess a similar loading regime in porcine tissues were made, however, due to difficulties with clamping the tissue to the mechanical tester and variable cross-sectional areas, reliable quantifiable data was not available to use as a comparison. Interestingly, it was observed that across all samples of porcine tissue (n=12) failure never occurred in the interfacial region. Thus, comparisons were made between the scaffold and reported values for native tissue in regions as similar to the scaffold as possible. In future studies, haemodynamic testing in a flow loop setting will ensure proper physiological loading of such a scaffold.

The ratio of strain rates in the circumferential to longitudinal direction was chosen as approximately 2:1. When tested to failure, the scaffold exhibited similar Young's modulus in both directions, whereas the yield strength in the circumferential direction was approximately double that of the longitudinal direction (see FIG. 13-2). Scaffold strength could be improved by adjusting layer number or fibre diameter. Strain-displacement vector mapping showed complex regional anisotropic deformation behaviour, where the circumferential and longitudinal strains were accounted for primarily by the leaflet and inter-leaflet triangle regions respectively. However, strain-displacement vectors showed a gradual change in direction and magnitude between these regions, indicating a smooth transition of load across the interface. Notably, the yield strain in the circumferential and longitudinal directions was 27.7±6.9% and 16.6±5.4% respectively, both of which were higher than the average maximum strains reported in literature across the different regions of the aortic valve. This implies the scaffold will remain within the elastic region and not plastically deform under physiological strains.

Hysteresis of the scaffold was then determined by cyclically testing the sample to a constant strain of 20% and 10% in the circumferential and radial directions, respectively, and calculating the ratio of the area under the unloading curve to the loading curve. The stress-strain plots are shown in FIG. 13-3, with both the plots for circumferential strain 1301 and longitudinal strain 1302. A constant low level of hysteresis (˜20% energy loss per cycle) was observed in both directions. Our data concurs with reported 17% hysteresis for porcine aortic heart valves in literature. The relaxation behaviour of the scaffold was characterized using 5% and 2.5% strain increments in the circumferential and radial directions, respectively, as shown in the FIG. 13-6. The scaffolds were then allowed to relax for 1000 seconds after each strain step, an adequate time frame to exhibit the majority of relaxation behaviour. The stress-time graph representing the relaxation behaviour is shown in FIG. 13-5. At all strain levels, the scaffolds exhibit rapid initial relaxation, before stabilising to an asymptote, which were then reported as relaxation percentages. A consistent degree of relaxation was maintained in the circumferential direction of 27.6±1.5%, whereas the radial direction exhibits initial large relaxation before stabilising to 30.6±1.7%. These values were remarkably similar to values reported for porcine aortic heart valve relaxation. In summary, the bio-inspired aortic heart valve interface scaffold thus demonstrated characteristics reminiscent of native tissue including anisotropy, hysteresis, as well as yield strains within that of native tissue.

In the above, a continuous interface across structural regions of a MEW scaffold is provided. Complex functions are utilised the define the print path between discrete end points in the structural regions connected by the interface region. Thus, the fibres take complex shapes as they transition from a region, through the interface region and to the next region which is structural to the first region. Simplified forms of the microstructural organisation of collagen fibres in the interface regions of biological tissue may be leveraged in designing the MEW tissue scaffold. For example, during testing and analysis conducted, the microstructural organisation of collagen fibres in the interface regions of the aortic heart valve was obtained using high resolution correlative multi-modal imaging. This was leveraged to inspire functional design of MEW heart valve scaffold including biomimetic interfacing regions for heart valve tissue engineering applications. A systematic morphological and mechanical study into three different methods of interfacing bi-phasic MEW scaffolds revealed that weaker regions dominate the tensile mechanical response. With regard to flexural stiffness, a property often overlooked in scaffold design, continuously interfacing bi-phasic scaffolds showed enhanced flexibility of the resulting constructs. Crucially, a novel software and accompanying GUI enabled the rapid design and G-code generation of not only an array of MEW complex scaffold designs, but also the continuous interfacial boundary across which these regions were connected. This software unlocks new capabilities of MEW for the field of interfacial tissue engineering. Subsequently, we designed, fabricated and tested a bio-inspired MEW scaffold for the aortic heart valve interfacial region, showing for the first time a scaffold containing continuous interfaces, gradient porosities, region-specific layer numbers and tailored fibre orientations in a singular biomimetic design. When tested under physiologically relevant bi-axial conditions, the scaffold exhibited promising tissue-like behaviour, including strain yield, hysteresis and relaxation all similar to native tissue.

### MEW Scaffold Fabrication

Scaffolds were fabricated using an in-house-built MEW device as reported previously, from medical-grade poly(ε-caprolactone) (PCL). The specific processing parameters used were: air pressure-driven extrusion at 100 kPa; through a 23G metallic needle; at a working distance of 3 mm; a translation speed of 400 mm/min; with voltage of 4.4±0.1 kV applied to the spinneret and grounded collector plate; with 86° C. ring heater and 31.5° C. bed heater. Exact processing parameters used were varied slightly depending on the environmental conditions on the day. All bi-phasic scaffolds used for tensile and flexural testing had dimensions of 10 mm height×40 mm width (20 mm width per pattern). This fabrication setting was found to allow fibre fusion within the same layer and also adequate bonding between different layers. Complete fusion between layers is in some cases not needed where the layers are not of identical configuration, or not desired depending on the degree of flexibility required for the overall layered structure.

### MEW Scaffold Imaging and Measurements

MEW scaffolds were first imaged using a Hirox RH-2000 digital microscope (Hirox, Europe) at a resolution of 4.51 μm/pixel. The accompanying software was used to measure fibre diameter at 3 locations across n=3 scaffolds of each type. Scaffolds were then cut using scissors and fixed to SEM stubs using double sided carbon tape. Stubs were then sputter coated with gold, two at a time, for 60 seconds, using a JEOL Smart Coater (JEOL, USA). SEM images were then acquired using a JCM-6000 Benchtop SEM (JEOL, USA) at acceleration voltage of 10 kV, 30× magnification, with high probe current and high vacuum mode.

### Uni-Axial Tensile Testing

Scaffolds were mechanically tensile tested uni-axially across the interface using the CellScale Biotester (CellScale, Waterloo, Canada) equipped with a 1.5N load cell. Samples (n=3 for each scaffold type) were secured with custom 3D printed miniature clamps and suspended in air at room temperature. Uni-axial tests used a displacement rate of 1% length/min. Stress-strain curves were plotted using the generated force-displacement data, where strain was defined as engineering strain (change in length/initial length). Initial length was kept constant at 15 mm for all calculations. Cross-sectional area was quantified as the scaffold wall height, as opposed to the peak height. Six measurements were taken across n=3 samples of each bi-phasic scaffold. Cross sectional area was observed to vary within individual scaffolds between the two patterns and the interface. However, as the majority of deformation occurs in the weaker pattern, confirmed by visual analysis, the cross-sectional area of the weaker pattern was used to calculate associated stresses. Averages for each pattern: serpentines=1.06±0.03 mm², diamonds=1.25±0.20 mm2, 1 mm squares=1.40±0.21 mm2, 0.5 mm squares=1.16±0.04 mm2. Young's modulus was calculated from the slope of the stress-strain curve at the steepest linear region, the strain range for which depended on the sample/pattern. Yield strengths were calculated as the point where the stress-strain curve plateaued. Ultimate tensile strength was determined as the maximum stress reached for each sample.

### Bi-Axial Tensile Testing

Bi-axial tensile tests were carried out using the same equipment as the uni-axial tests, with a 5N load cell. A pre-load of 50 mN was applied before every test to ensure a consistent initial state of the scaffolds. Young's modulus and yields strength were calculated using the same protocol as for uni-axial tests. Cyclical testing was conducted for 1 preload cycle (to establish the in-vivo state of the material) and 9 subsequent loading cycles, which were then plotted as stress-strain. Hysteresis was calculated as the ratio of the area under the unloading curve to the loading curve for each cycle. The relaxation behaviour of the scaffold was characterized using 5% and 2.5% strain increments in the circumferential and radial directions respectively (FIG. 8D). The scaffolds were then allowed to relax for 1000 seconds, an adequate time frame to exhibit the majority of relaxation behavior. Relaxation percentages were reported as the difference between maximum and minimum stress for each step.

### Flexural Testing

Flexural testing was conducted using the Peirce cantilever test method (ASTM D1388). A custom testing apparatus was designed in Fusion 360 (Autodesk, USA) and printed using Prusament PLA (Prusa Research, Czech Republic) with a Prusa MK3s+ (Prusa Research, Czech Republic). Angles were measured 6 times independently per flexing pattern, thrice in one direction and then thrice more with the scaffolds flipped over to account for the natural warping of the scaffold after printing.

### Complex G-Code Generator GUI and Path Visualisation

An in-house program was developed using Python (Python Software Foundation, USA) to incorporate continuous interface design into spatially heterogeneous G-codes for MEW printing. A script generating a GUI was made, for which the desired parameters of the scaffold could be entered. Once entered the interface would execute other scripts that perform the necessary calculations to plot data for the desired printing path. This data was then converted by another script into a G-Code Notepad file for input into MEW printer software. To permit visualization of G-code, another custom Python based software was built that imports raw G-code and produces both stationary and dynamic renders of the print path, with customizable scale, colours, line widths and path speeds.

### Orientation Mapping

Orientation analysis of SHG images and MEW scaffolds was done using the plugin OrientationJ [90], for the software Fiji (National Institute of Health, USA). [91] Using the OrientationJ Analysis module, a hue-saturation-brightness colour survey was calculated using a 2-pixel cubic spine. Then the OrientationJ Distribution module was used to plot the distribution of orientations.

### Statistical Analysis

All data was presented as mean±standard deviation for n=3 samples unless stated otherwise. For uni-axial tensile testing data, significant differences were assessed using one-way ANOVA with Tukey's multiple comparisons test. For flexural stiffness data, significant differences were assessed using two-way ANOVA with Tukey's multiple comparisons test. For bi-axial tensile testing data, significant differences were assessed using parametric, ratio paired t tests. Alpha was 0.05 for all tests. All statistical analysis was conducted using GraphPad Prism software (GraphPad, San Diego, USA).

As mentioned, the MEW printing described above contrasts with conventional MEW printing, where a layer comprising separate structural regions would not be printed in one continuous run. Rather, the regions of the layer would be separately printed and then sutured to form the layer. The mechanical advantages of continuous printing through the regions were not envisaged. The contrast with conventional thinking is even greater in embodiments where the fibres are purposely made to converge into a thicker fibre. This is considered “fibre bridging” which in conventional wisdom has always been avoided as a defect. This is because in MEW, there is a limit to the ability to make the pore size small, due to the electrostatic fibre repulsion from the entrapped charges inherent to the MEW printing process, where the high voltage electrostatically charges the molten jet, and when it cools down, the charges get entrapped. This phenomenon is still being understood by persons skilled in the art, who would be expected to consider the convergence of fibres to be something which is usually avoided. However, in some embodiments of the present method, “fibre bridging” is purposefully caused in a controlled manner. The MEW printing parameters presented above allow control of the bonding between the two merging fibres to become one single thicker fibre. This opens up possibilities of creating biomimetic designs for the MEW scaffold, utilising data regarding fibre distribution which can be obtained using imaging modalities. Scaffolds designed using this methodology can be used for the fabrication of soft tissue implants with biodegradable materials, or for the fabrication of non-biodegradable implantable devices.

### Example 6

### Gradient Porosity Scaffolds

G-code is the language that most 3D printers use, which specifies cartesian (xyz) coordinates for the printer to move to in a particular order. A G-code generator was made using Python that allows the user to input desired design parameters and produce an output G-code to control the MEW printer as well as an image preview of the scaffold architecture. The novelty of this generator is the ability to specify a gradient change in porosities.

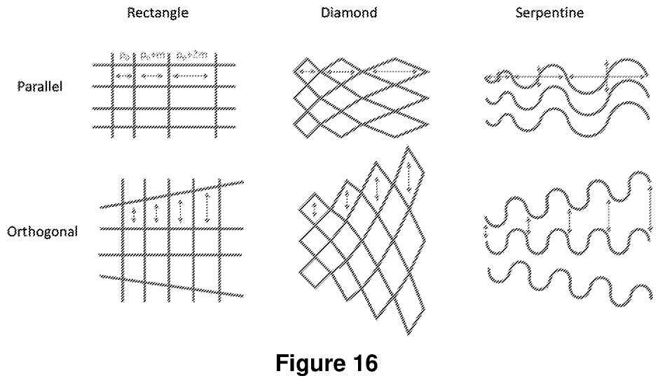

The gradient is mathematically defined and can be customised (FIG. 16). The gradient function is a linear function determined by:

$${p}_{n + 1} = {p}_{n} + m$$

Where pₙ is the pore size (any positive number in mm) and m is the gradient coefficient (any non-zero number, where positive m will increase the pore size and negative m will decrease it).

Since there are two dimensions to every pore (length (X) and width (Y)), the gradient can be applied to either one or both of these dimensions. These are referred to as either an X-gradient or a Y-gradient respectively, indicating the pore dimension that is changing.

The overall scaffold architecture itself is also two-dimensional, and thus the pore size can be changed in either one or both of these directions (horizontally or vertically). For simplicity, in this example the gradient is only applied in the horizontal direction.

This gives two types of gradients:
X-gradient, applied horizontally. This can be seen as changing the pore size that is parallel to the direction of the gradient, and thus is referred to as a parallel gradient.
Y-gradient, applied horizontally. This can be seen as changing the pore size that is orthogonal to the direction of the gradient, and thus is referred to as an orthogonal gradient.

Here, gradients were applied to three different patterns/shapes (rectangle, diamond or serpentine). Note that in the case of the serpentine scaffolds the wavelength, amplitude, or both can be varied.

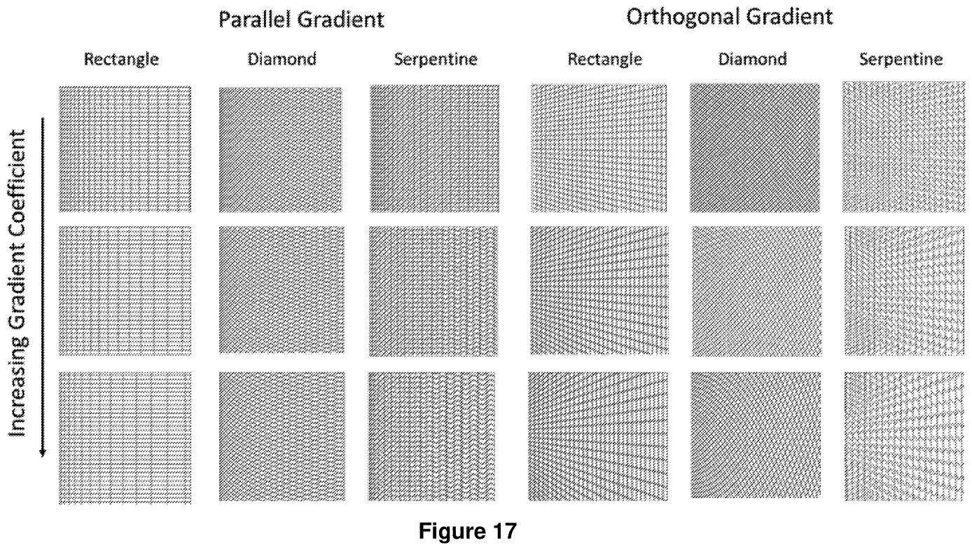

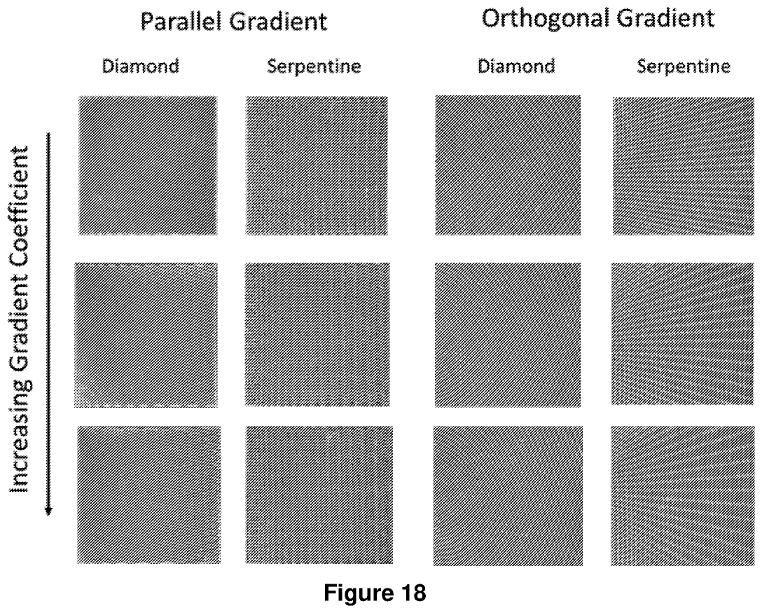

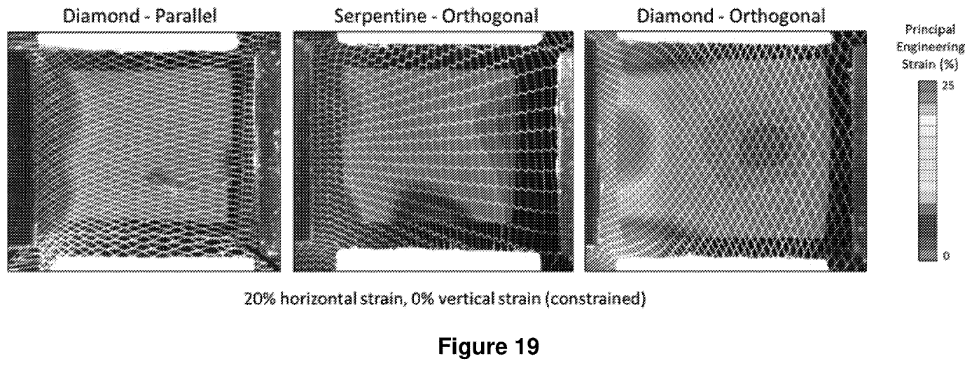

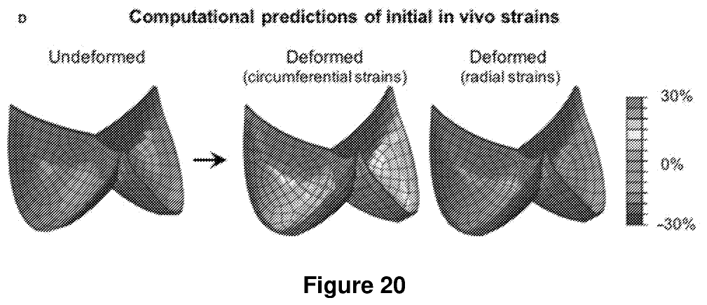

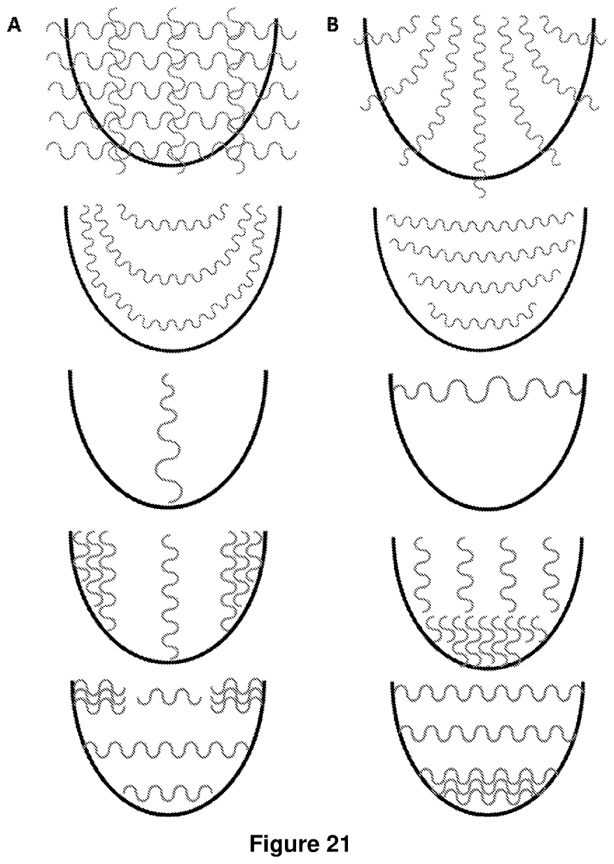

By gradient it is meant a gradual spatial change in properties (geometric features, mechanical properties, composition, chemistry or more). The present scaffolds create multiple gradient properties in a single scaffold. Geometrically this is already evident (FIGS. 16-19), however there are also gradient mechanical properties (FIG. 20). Note that while not shown here, other properties such as gradient tissue/cell composition may potentially be enabled/achieved through the use of these scaffolds.

To understand the effect of gradients on mechanical properties, constrained bi-axial tensile testing was conducted. This is where the scaffold is clamped on its four edges and then pulled in the horizontal direction, while being held (constrained) in the vertical direction. This allows observation of the local changes in strain throughout the scaffold, strain being how much deformation occurs in an object. FIG. 20 demonstrates the highly heterogeneous mechanical properties produced as a result of gradient scaffold architectures. Note that a completely uniform/homogeneous scaffold design would show the same colour all throughout. The novelty here is the ability to create a gradient of mechanical properties throughout a single scaffold. The present invention provides the ability to create gradients not just in one direction, but in complex arrangements, which may have benefits for tissue-relevant applications.

Variations and modifications may be made to the parts previously described without departing from the spirit or ambit of the disclosure.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of the inventors' contribution. The actual scope of the protection sought is intended to be defined in the following claims when viewed in their proper perspective based on the prior art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A melt electrowritten soft tissue scaffold, comprising:
a first region having one or more sets of fibres and a second region having one or more sets of fibres; and
an interface region joining the first region and the second region, the interface region being electrowritten along with the first and second regions in a continuous printing path, such that fibres in the interface region each join between a respective pair of fibres in the first and second regions.

2. The scaffold of claim 1, wherein a path for at least one of the fibres within the interface region is defined manually or defined by a mathematical function.

3. The scaffold of claim 2, wherein the function is defined such that the fibre has a complex shape as it transitions from the one of the first or second regions through the interface region and to the other one of the first or second regions.

4. The scaffold of claim 1, wherein the second region has a higher porosity than the first region, or vice versa.

5. The scaffold of claim 4, wherein the higher porosity in the second region is at least partially formed by joining every two or more adjacent fibres in the first region as they transition to the second region.

6. The scaffold of claim 4, wherein the higher porosity in the second region is at least partially formed by causing the fibres to fan outwards as they transition to the second region.

7. The scaffold of claim 4, wherein the higher porosity in the second region is at least partially formed by setting the continuous printing path to cause an additional set of fibres to be deposited that are offset from the other fibres in the first region by a distance which is less than the size of a pore size.

8. The scaffold of claim 1, wherein the first region or the second region, or both, includes a first set of fibres arranged approximately in parallel to each other and a second set of fibres arranged approximately in parallel to each other, the second set of fibres being arranged at an angle and preferably transversely relative to the first set of fibres, each fibre in the second set of fibres having a serpentine arrangement having defined troughs and peaks.

9. The scaffold of claim 1, wherein the first and second regions are heterogeneous in that they differ from each other in one or more spatial parameters.

10. The scaffold of claim 1, comprising a plurality of layers of fibres.

11. The scaffold of claim 10, having a portion which has a different number of layers than a different portion.

12. The scaffold of claim 1, being a heart valve scaffold.

13. The scaffold of claim 12, wherein the first region is a leaflet and the second region is an inter-leaflet triangle.

14. A method of providing a melt electrowritten soft tissue scaffold having at least two structural heterogeneous regions and an interface region there between, including providing a printing path along which polymer melt material is to be continuously extruded during a melt electrowriting process to form the heterogeneous structural regions and the interface region in one printing run.

15. The method of claim 14, including causing the continuous print path to be piece-wise defined such that it is defined by different functions in the heterogeneous regions and in the interface region, respectively.

16. The method of claim 15, wherein function or functions which define the continuous print path as it traverses through the interface region is a mathematical function providing a curved or serpentine shape.

17. The scaffold of claim 1, comprising gradient porosities, wherein the size of the pores gradually increases or decreases in either one direction, or two directions spatially across the scaffold.

18. The scaffold of claim 17, wherein the shape of the pore within the scaffold is chosen from: rectangular, diamond or serpentine.

19. The scaffold of claim 1, wherein the scaffold comprises a plurality of layers of fibres.

20. The scaffold of claim 19 wherein the plurality of layers of fibres comprises gradients of geometric features and/or mechanical properties.

21. The scaffold of claim 17, wherein the scaffold comprising gradient porosities is used for the manufacture of heart valves.
